(19) United States
(12) Patent Application Publication
(10) Pub. No.: US 2025/0331757 A1
(43) Pub. Date: Oct. 30, 2025

(54) ELECTRONIC DEVICES AND METHODS THEREOF

(71) Applicant: PEKING UNIVERSITY, Beijing (CN)

(72) Inventors: Xiaojie DUAN, Beijing (CN); Yang LIU, Beijing (CN); Huilin JIA, Beijing (CN); Anqi JIANG, Beijing (CN); Hongji SUN, Beijing (CN); Ziqian YANG, Beijing (CN); Shengyi JIA, Beijing (CN)

(73) Assignee: PEKING UNIVERSITY, Beijing (CN)

(21) Appl. No.: 19/261,198

(22) Filed: Jul. 7, 2025

Related U.S. Application Data
(63) Continuation of application No. PCT/CN2024/070992, filed on Jan. 6, 2024.

(30) Foreign Application Priority Data
Jan. 6, 2023 (CN) 202310019748.5
Jan. 6, 2023 (WO) PCT/CN2023/071106

(51) Int. Cl. A61B 5/293; A61B 5/03

(52) U.S. Cl. CPC A61B 5/293; A61B 5/031; A61B 2560/0406; A61B 2560/0468; A61B 2562/0247; A61B 2562/046; A61B 2562/125; A61B 2562/227

(57) ABSTRACT

The present disclosure relates to an electronic device and a method thereof. The device may include a target electrode array having a plurality of electrode sites arranged circumferentially around a surface of the device and/or axially along the surface of the device. The device may be formed by scrolling and/or attaching a planar electrode array around a carrier.

### CROSS-REFERENCE TO RELATED APPLICATIONS

This application is a continuation of International Application No. PCT/CN2024/070992, filed on Jan. 6, 2024, which claims priority to International Application No. PCT/CN2023/071106, filed on Jan. 6, 2023, and Chinese Patent Application No. 202310019748.5, filed on Jan. 6, 2023, the contents of each of which are hereby incorporated by reference.

### TECHNICAL FIELD

The present disclosure generally relates to the field of electronics technology, including the implantable bioelectronics technology, and more particularly, relates to electronic and bioelectronic devices and methods thereof.

### BACKGROUND

Sensory, motor, and cognitive operations involve the coordinated action of large neuronal populations across multiple brain regions in both superficial and deep structures. Techniques with high-throughput and high spatial-temporal resolution facilitate the study of large scale and dynamic neural networks to understand the neural circuit mechanisms and modulate neural activities in neurological diseases. Extracellular neural electrodes with high spatial-temporal resolution can detect single neuron activities. Neural electrodes generated based on complementary metal-oxide semiconductor (CMOS) silicon fabrication technology have high throughput for neuronal signal detection, but the inherent rigidity makes them mechanically incompatible with neural tissues and easy to break, and thus, it may be difficult for the CMOS-based silicon neural electrodes to reach deep brain regions in large animal models or humans. Existing flexible neural electrodes generated based on polymer film fabrication technology have biocompatibility and long-term stability, but a shuttle is required for an implantation process. The implantation of the flexible neural electrodes into the subcortical deep brain regions remains a challenge. Limited longitudinal dimensions of the existing flexible neural electrodes further make it difficult for them to reach deep brain regions. In addition, the preparation and performance of the flexible neural electrodes are restricted by the complexity of micro/nano fabrication.

Thus, it is desirable to provide an implantable device, which has high density and throughput, is adjustable in length, and is mechanically stable, unbreakable, maneuverable, to facilitate its application to a wide range of organisms.

Besides, in fields such as integrated circuits, bioelectronics, and neural engineering, connections between electronic components are essential. However, traditional soldering techniques applied to electrical components impose relatively strict requirements on external conditions (e.g., temperature, pressure, etc.), electrical components connected using traditional soldering techniques are difficult to be separated, and separate electrical components are difficult to be connected again. Therefore, it is desirable to provide a simple, easy-to-operate, and reversible method for connecting electronic components.

### SUMMARY

According to an aspect of the present disclosure, a device may be provided. The device may include a target electrode array having a plurality of electrode sites arranged circumferentially around a surface of the device and/or axially along the surface of the device. The device may be formed by rolling and/or attaching a planar electrode array around a carrier.

In some embodiments, the planar electrode array may include an electrode site part, a lead part, and an interface part. The plurality of electrode sites of the target electrode array may be arranged in the electrode site part. The lead part may be configured to connect the electrode site part to the interface part. The interface part may be configured to connect the device to a second device.

In some embodiments, after the planar electrode array is scrolled and/or attached around the carrier, the electrode site part may be exposed on a surface of the device, the lead part may be embedded in an internal part of the device or exposed on a surface of the device, and the interface part may be connected to the lead part.

In some embodiments, the planar electrode array may include a connection part for connection to the carrier.

In some embodiments, the connection part may include a hole structure or a mesh structure, such that the carrier passes through the hole structure or the mesh structure to be fixed on the connection part.

In some embodiments, the carrier may be bonded to the connection part of the planar electrode array via an adhesive material.

In some embodiments, the carrier may be fixed on the connection part of the planar electrode array by putting the carrier into a groove on the planar electrode array.

In some embodiments, the planar electrode array may be connected to the carrier after the planar electrode array is released from a substrate.

In some embodiments, the planar electrode array may be connected to the carrier before the planar electrode array is released from a substrate.

In some embodiments, the lead part may be of a “U” shape, the lead part may be arranged on an opposite end to that of the electrode site part and the interface part on the planar electrode array.

In some embodiments, the lead part may be arranged on the two sides of the electrode site part.

In some embodiments, the carrier and the planar electrode array may be configured as an integral piece.

In some embodiments, a side of the planar electrode array may have a bevel edge structure. The bevel edge structure may be configured to form a conical structure on the device by rolling the planar electrode array around the carrier.

In some embodiments, the conical structure may be at a tip of the device.

In some embodiments, at least one of a substrate or an encapsulated layer of the planar electrode array may be made of a flexible material.

In some embodiments, the flexible material may include at least one of polyimide, Parylene-C, polydimethylsiloxane (PDMS), polystyrene-block-poly (ethylene-ran-butylene)-block-polystyrene (SEBS), SU-8 photoresist, polyethylene terephthalate, polyurethane, Ecoflex, poly(styrene-butadiene-styrene), or Teflon.

In some embodiments, a thickness of the planar electrode array may be in a range of 50-100000 nanometers.

In some embodiments, a conducting layer of the target electrode array may be made of at least one of gold, platinum, copper, aluminum, silver, titanium, chromium, nickel, tantalum, palladium, molybdenum, a carbon nanotube (CNT), a graphene, a carbon material, iridium oxide, titanium nitride, a conducting polymer, indium tin oxide, tantalum oxide, or a liquid metal.

In some embodiments, a length of the target electrode array along a longitudinal axis of the device may be in a range of several millimeters to tens of centimeters.

In some embodiments, a count of the plurality of electrode sites of the target electrode array may be in a range of 1-50000.

In some embodiments, the carrier may be configured to scroll the planar electrode array, improve a mechanical strength of the device, perform an optical stimulation or an electrical stimulation on a subject, or perform a delivery of drugs and/or reagents.

In some embodiments, the carrier may include at least one of a metal wire, a metal tube, a quartz wire, a quartz tube, an optical fiber, a polymer wire, a polymer tube, a combination of wire and tube, a ceramic wire, a graphene fiber, a carbon fiber, a stereoelectroencephalography (SEEG) electrode, or an intracranial pressure measuring device.

In some embodiments, a length of the carrier may be in a range of several millimeters to tens of centimeters, and/or a diameter of the carrier may be in a range of 5-2000 micrometers.

In some embodiments, a length of the device along a longitudinal axis of the carrier may be in a range of several millimeters to tens of centimeters, and/or a diameter of the device may be in a range of 10-3000 micrometers.

In some embodiments, a size of an electrode site of the target electrode array may be in a range of 1-3000 μm.

According to another aspect of the present disclosure, a method for preparing a device may be provided. The method may include preparing a planar electrode array. The method may include rolling the planar electrode array around a carrier to form the device. The device may have a columnar structure.

In some embodiments, the method may include preparing a nickel-deposited silicon wafer by depositing nickel on a silicon wafer. The nickel may be determined as a sacrificial layer of the planar electrode array. The method may include preparing a substrate of the planar electrode array by spin coating, heating, and curing at least one of polyamic acid, polydimethylsiloxane (PDMS), SU-8 photoresist, or by depositing Parylene-C, on the nickel-deposited silicon wafer. The method may include preparing a conductive layer of the planar electrode array by depositing and patterning at least one of a metal, a carbon nanotube, a graphene, a carbon material, iridium oxide, titanium nitride, indium tin oxide, tantalum oxide, or a conductive polymer on the substrate of the planar electrode array. The method may include releasing the planar electrode array by etching the sacrificial layer from the silicon wafer using ferric chloride solution.

In some embodiments, after the preparing a conductive layer of the planar electrode array, the method may include preparing an encapsulated layer of the planar electrode array by spin coating, heating, and curing the at least one of polyamic acid, PDMS, SU-8 photoresist, or by depositing Parylene-C, on the conductive layer of the planar electrode array. The method may include preparing a mask by depositing and patterning aluminium on the encapsulated layer of the planar electrode array to obtain a masked encapsulated layer. The method may include obtaining a patterned electrode array by processing the masked encapsulated layer using reactive ion etching. The method may include releasing the planar electrode array by etching the mask and the sacrificial layer from the silicon wafer using the ferric chloride solution.

In some embodiments, the method may include fixing the carrier on a first side of the planar electrode array. The method may include scrolling and/or attaching the planar electrode array around the carrier. The method may include coating, in a last turn of the scrolling and/or attaching, an edge of a second side of the planar electrode array with an adhesive material, to fix the second side of the planar electrode array on the device.

In some embodiments, the adhesive material may include at least one of a polyethylene oxide (PEO) solution, a polyethylene glycol (PEG) solution, a silk fibroin solution, a Kollicoat® solution, a biological glue, a medical glue, a sucrose solution, a gelatin, or a photoresist.

In some embodiments, the method may further include coating, during the scrolling and/or attaching, at least a portion of a rear surface of the planar electrode array with the adhesive material, to fix the rear surface of the planar electrode array on the device.

In some embodiments, the method may include fixing the carrier on the first side of the planar electrode array via a connection part of the planar electrode array.

In some embodiments, the connection part may include a hole structure or a mesh structure, such that the carrier passes through the hole structure or the mesh structure to be fixed on the connection part.

In some embodiments, the method may include bonding the carrier to the connection part of the planar electrode array via an adhesive material.

In some embodiments, the method may include fixing the carrier on the connection part of the planar electrode array by putting the carrier into a groove on the planar electrode array.

In some embodiments, the carrier and the planar electrode array may be configured as an integral piece.

In some embodiments, the carrier may be capable of being fully or partly removed from or left with the device.

In some embodiments, the method may further include: connecting the device to a second device.

In some embodiments, the device may include a first connection site, the second device may include a second connection site, and the connecting the device to a second device may include aligning the first connection site of the device with the second connection site of the second device in a solution environment; and drying the first connection site and the second connection site, such that a physically electrically conductive connection is formed between the aligned first connection site and second connection site.

According to another aspect of the present disclosure, a method for connecting electronic components is provided. The method may include: aligning a first connection site of a first electronic component with a second connection site of a second electronic component in a solution environment; and drying the first connection site and the second connection site, such that a physically electrically conductive connection is formed between the aligned first connection site and second connection site.

In some embodiments, the first electronic component may include a flexible electronic component; and the first connection site may include a first conductive structure.

In some embodiments, a thickness of the first connection site may be of a micrometer or submicrometer scale.

In some embodiments, the second electronic component may include at least one of a printed circuit board, a flexible flat cable, a flexible circuit board, a micromachined chip, a complementary metal oxide semiconductor (CMOS) chip, an integrated circuit (e.g., an application specific integrated circuit), or a CMOS metal microelectrode array; and the second connection site may include a second conductive structure.

In some embodiments, the first connection site may have a mesh structure.

In some embodiments, the second connection site may have a mesh structure.

In some embodiments, the physically electrically conductive connection formed between the first connection site and the second connection site may make the first connection site and the second connection site be electrically conductive to each other; and a contact resistance between the first connection site and the second connection site may be less than or equal to 10 MΩ.

In some embodiments, a connection area between the first connection site and the second connection site may be greater than or equal to 0.1 square micrometers.

In some embodiments, the drying the first connection site and the second connection site may include: drying the first connection site and the second connection site such that the first connection site is bent to the second connection site under a surface tension to form the physically electrically conductive connection.

In some embodiments, the first electronic component may include a first substrate, the second electronic component may include a second substrate, and the method may further include: drying the first substrate and the second substrate such that a physical adsorption is formed between the first substrate and the second substrate.

In some embodiments, the method may further include: separating the physically electrically conductive connection formed between the first connection site and the second connection site in the solution environment; and re-aligning and re-drying the separated first connection site and second connection site, to reform the physically electrically conductive connection between the first connection site and the second connection site.

In some embodiments, the first electronic component may include a device, and the device may include: a target electrode array having a plurality of electrode sites arranged circumferentially around a surface of the device and/or axially along the surface of the device, wherein the device is formed by rolling and/or attaching a planar electrode array around a carrier.

In some embodiments, the second electronic component may include at least one of a printed circuit board (PCB), a flexible flat cable (FFC), a flexible extender board, or an integrated circuit chip.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIGS. 1A-1B are schematic diagrams illustrating an exemplary planar electrode array according to some embodiments of the present disclosure;

FIG. 1C is a picture of an exemplary planar electrode array on a nickel-deposited silicon wafer according to some embodiments of the present disclosure;

FIG. 2A is a schematic diagram illustrating an exemplary connection between a planar electrode array and a carrier according to some embodiments of the present disclosure;

FIG. 2B is a schematic diagram illustrating an exemplary scrolling process of a planar electrode array around a carrier according to some embodiments of the present disclosure;

FIG. 2C is a schematic diagram illustrating an exemplary device according to some embodiments of the present disclosure;

FIG. 2D is a picture illustrating an exemplary connection part according to some embodiments of the present disclosure;

FIG. 3 is a micrograph picture illustrating an exemplary distribution of a plurality of electrode sites in a target electrode array according to some embodiments of the present disclosure;

FIG. 4A is a picture illustrating an exemplary connection between a planar electrode array and a carrier according to some embodiments of the present disclosure;

FIG. 4B is a picture of a device according to some embodiments of the present disclosure;

FIGS. 5A and 5B are schematic diagrams illustrating an exemplary connection between a device and a second device according to some embodiments of the present disclosure;

FIGS. 5C and 5D are pictures illustrating an exemplary connection between a device and a second device according to some embodiments of the present disclosure;

FIG. 6 is a flowchart illustrating an exemplary process for preparing a device according to some embodiments of the present disclosure;

FIG. 7 is a schematic diagram illustrating an exemplary process for preparing a planar electrode array according to some embodiments of the present disclosure;

FIG. 8 is a flowchart illustrating an exemplary process for implanting a device into a subject according to some embodiments of the present disclosure;

FIG. 9A is a picture illustrating an exemplary process for implanting a device into the brain of a rat according to some embodiments of the present disclosure;

FIG. 9B is an enlarged view of a region 910 shown in FIG. 9A according to some embodiments of the present disclosure;

FIG. 9C is a picture illustrating neural signals recorded in a rat according to some embodiments of the present disclosure;

FIG. 10A is a picture illustrating an exemplary process for implanting a device into the brain of a rhesus monkey according to some embodiments of the present disclosure;

FIG. 10B is a picture illustrating neural signals recorded in a rhesus monkey according to some embodiments of the present disclosure;

FIG. 11 is a flowchart illustrating an exemplary process for connecting electronic components according to some embodiments of the present disclosure;

FIG. 12 is a schematic diagram illustrating an exemplary process for aligning electronic components to be connected according to some embodiments of the present disclosure;

FIG. 13 is a schematic diagram illustrating an exemplary structure of a first connection site of a first electronic component according to some embodiments of the present disclosure;

FIGS. 14A and 14B are schematic diagrams illustrating an exemplary connection effect of a first electronic component and a printed circuit board according to some embodiments of the present disclosure;

FIG. 15 is an exemplary waveform illustrating an application of connected electronic components in signal detection after the first electronic component is connected to the printed circuit board according to some embodiments of the present disclosure;

FIG. 16 is a schematic diagram illustrating an exemplary connection effect of a first electronic component and a flexible flat cable according to some embodiments of the present disclosure; and

FIGS. 17A and 17B are schematic diagrams illustrating an exemplary structure of the first electronic component connected with a flexible circuit board according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

As indicated in the present disclosure and claims, unless the context clearly suggests an exception, the words “one,” “a,” “a kind of,” and/or “the” do not specifically refer to the singular, but may include the plural. Generally, the terms “including” and “comprising” suggest only the inclusion of clearly identified steps and elements, which do not constitute an exclusive list, and the method or apparatus may also contain other steps or elements. The term “based on” is “at least partially based on”. The term “one embodiment” means “at least one embodiment”; the term “another embodiment” means “at least one additional embodiment”.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

In the present disclosure, the terms “connected”, “linked”, “fixed”, etc. are to be understood in a broad sense, unless otherwise expressly specified and qualified. For example, it may be a fixed connection, a detachable connection, or a one-piece connection; it may be a mechanical connection or an electrical connection; it may be a direct connection or an indirect connection through an intermediate medium, a connection between two elements or an interactive relationship between two elements, unless otherwise expressly limited. For those skilled in the art, the specific meaning of the above terms in the present disclosure may be understood on a case-by-case basis.

In the description of the present disclosure, it should be understood that the terms “upper”, “lower”, “surface” etc. indicate an orientation or positional relationship based on that shown in the accompanying drawings, and are intended only to facilitate the description of the present disclosure and to simplify the description, and are not intended to indicate or imply that the device or element referred to must be of a particular orientation, be constructed and operated with a particular orientation, and therefore are not to be construed as a limitation of the present disclosure.

Furthermore, the terms “first” and “second” are used for descriptive purposes only and are not to be understood as indicating or implying relative importance or implicitly specifying the number of technical features indicated. Thus, a feature defined with “first” or “second” may include at least one such feature, either explicitly or implicitly. In the description of the present disclosure, “plurality” means at least two, such as two, three, etc., unless otherwise expressly and specifically limited.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

An aspect of the present disclosure relates to an implantable device. The device (e.g., a bioelectronic probe) may include a target electrode array. The target electrode array may have a plurality of electrode sites arranged circumferentially around a surface of the device and/or axially along the surface of the device. The device may be formed by scrolling and/or attaching a planar electrode array around a carrier. In some embodiments, after the planar electrode array is scrolled and/or attached around the carrier to form the device, the carrier may be fully or partly left with the device or removed from the device.

According to some embodiments of the present disclosure, the device (e.g., the bioelectronic probe) with dense recording/stimulating sites and a high count of channels may be provided. The device may be used for recording and/or stimulating physiological activities (e.g., bioelectrical activities like neural activities). A size (e.g., a length) of the device may be adjustable in a large range (e.g., several millimeters to several tens of centimeters) based on actual requirements, so that the device may be applied to a wide range of organisms and organs, such as a plurality of brain regions of the brain, and the cerebral cortex, deep subcortical areas, of a primate and/or a rodent. In addition, an arrangement of the electrode sites (e.g., a count of the electrode sites, a position of the electrode site, a distance between adjacent electrode sites) of the device may be designed according to actual requirements. The electrode sites may be modified or adjusted to realize electrophysiological and/or neurochemical recording, and/or electrical stimulation in multiple brain regions. Furthermore, the device may be fabricated by a micro/nano fabrication process, which may be simple, reliable, and of strong operability. The device may have high mechanical stability, and be not easy to break. Accordingly, the device may have a wide range of applications (e.g., from a single brain region of a small animal model to multiple brain regions of a primate), and can realize the detection of the cerebral neural circuit.

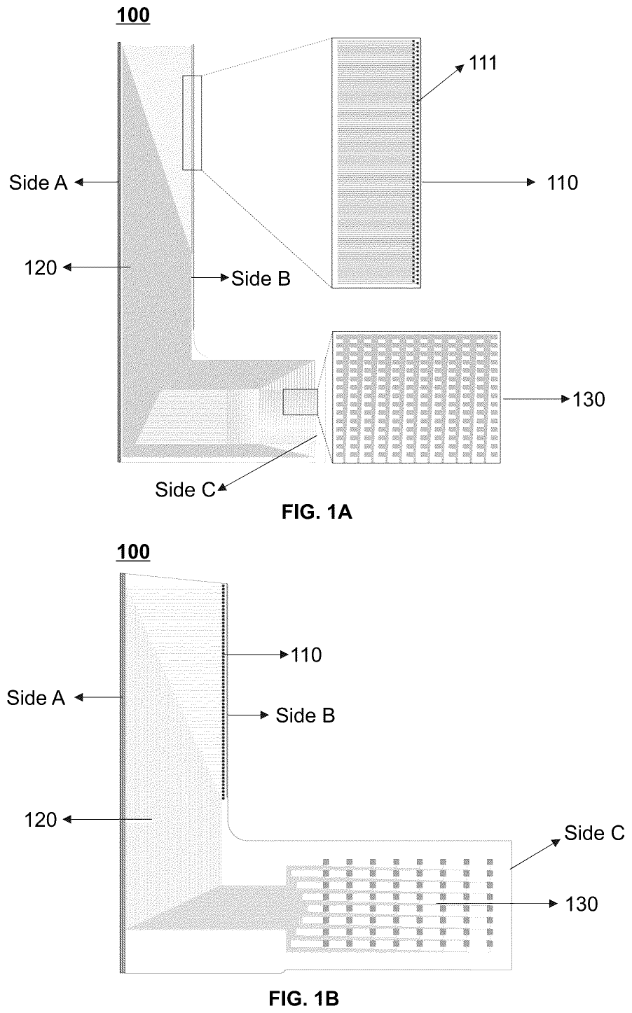

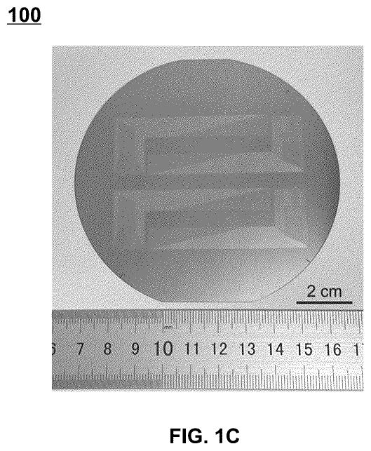

FIGS. 1A-1B are schematic diagrams illustrating an exemplary planar electrode array according to some embodiments of the present disclosure. FIG. 1C is a picture of an exemplary planar electrode array on a nickel-deposited silicon wafer according to some embodiments of the present disclosure.

A planar electrode array 100 may be used in preparing a device. For example, the planar electrode array 100 may be scrolled and/or attached around a carrier to form the device. In some embodiments, after the planar electrode array 100 is scrolled and/or attached around the carrier to form the device, the carrier may be fully or partly left with the device or removed from the device. In some embodiments, the planar electrode array 100 may be a thin flexible planar electrode array. For example, a thickness of the planar electrode array 100 may be in a range of 50-100000 nanometers. In some embodiments, the thickness of different regions of the planar electrode array 100 may be the same or different.

As illustrated in FIGS. 1A and 1B, the planar electrode array 100 may include an electrode site part 110, a lead part 120, and an interface part 130. In some embodiments, as illustrated in FIGS. 1A and 1B, the lead part 120 may be located at or close to a first side (e.g., side A) of the planar electrode array 100, the electrode site part 110 may be located at a second side (e.g., side B) of the planar electrode array 100, and the interface part 130 may be located at a third side (e.g., side C) of the planar electrode array 100.

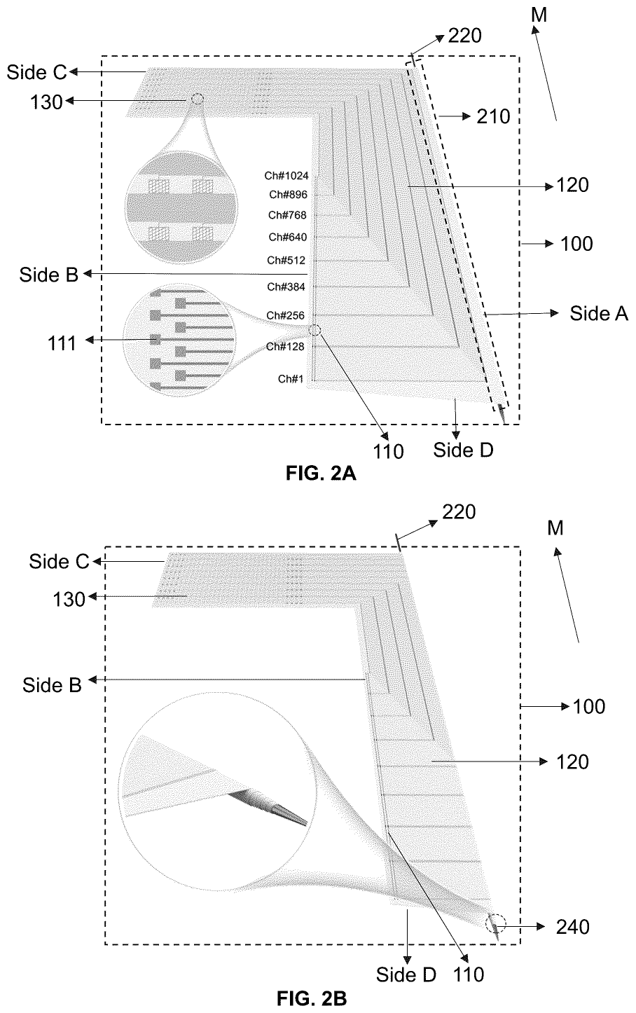

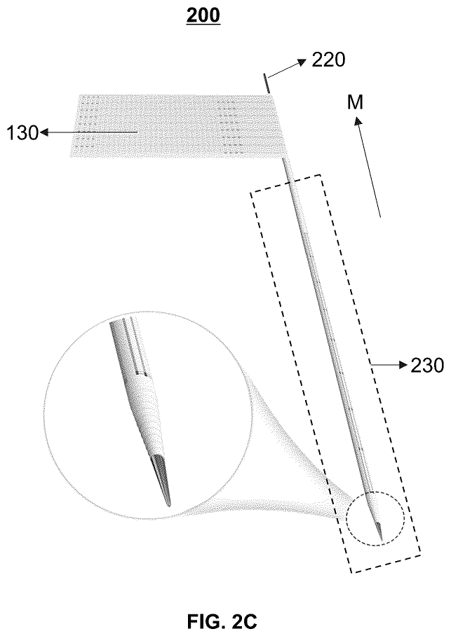

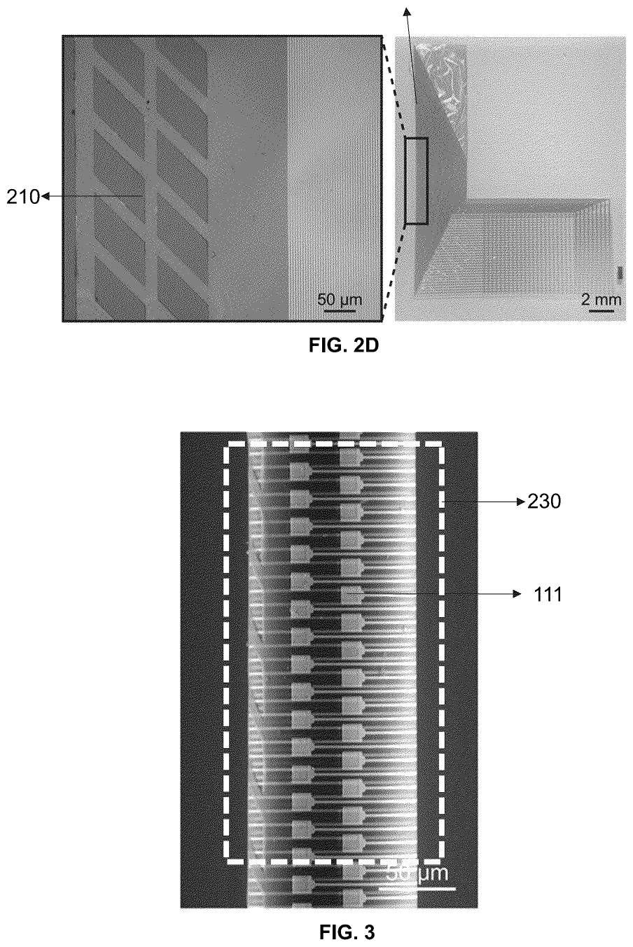

The electrode site part 110 may include a plurality of electrode sites (e.g., a plurality of electrode sites 111 as illustrated in FIG. 1A). The plurality of electrode sites may form the target electrode array (e.g., a target electrode array 230 as illustrated in FIG. 2C and FIG. 3) of the device as described elsewhere the present disclosure. The electrode sites may be in electrical communication with a subject that the electrode sites coupled to or implanted in to detect electrophysiological activities and/or parameters (e.g., physiological parameters) of the subject. The subject may include a patient, an animal, or the like, or a specific portion (e.g., an organ, tissue) thereof. In some embodiments, the plurality of electrode sites in the electrode site part 110 may be of the same type or of different types. In some embodiments, one or more electrode sites of the plurality of electrode sites may be activated to detect electrophysiological activities and/or parameters of the subject according to actual requirements, and the other electrode sites may be inactivated. In some embodiments, surfaces of one or more electrode sites of the plurality of electrode sites may be chemically or biologically modified to expand the detection capability of the device. For example, modified electrode sites may be used for electrochemical detection to detect physiological parameters of the subject, and/or injecting electrical pulses to modulate the activity of the subject.

In some embodiments, the electrode site part 110 may include any number (or count) of electrode sites. For example, the electrode site part 110 may include dozens, hundreds, thousands or tens of thousands of electrode sites. In some embodiments, the plurality of electrode sites may be arranged at any positions in the electrode site part 110 in any suitable manner. For example, the plurality of electrode sites may be arranged in an array. The array may include a plurality of columns of electrode sites and a plurality of rows of electrode sites. Different columns or rows may include the same number (or count) or different numbers (or counts) of electrode sites. As illustrated in FIG. 1A, the plurality of electrode sites may be arranged in a staggered array. In some embodiments, the array may be of a planar shape. In some embodiments, the plurality of electrode sites may be arranged in a plurality of arrays. The plurality of arrays may be arranged in a same layer or different layers of the planar electrode array 100.

In some embodiments, the arrangement density of the electrode sites in the electrode site part 110 may be uniform or non-uniform. For example, a distance between adjacent electrode sites of the plurality of electrode sites in the electrode site part 110 may be the same or different.

The lead part 120 may be configured to connect the electrode site part 110 to the interface part 130, so as to conduct electrical signals between the electrode site part 110 and the interface part 130. In some embodiments, the lead part 120 may include a plurality of wires or lines. The plurality of wires or lines may transmit a plurality of channels of signals (e.g., a bioelectrical signal, a control signal, an excitation signal, and/or signals relating to information (e.g., a physiological parameter)) between the electrode site part 110 and the interface part 130. In some embodiments, the wires may include a metal wire or metal line. In some embodiments, the interface part 130 may include a plurality of pads. Each electrode site may be connected to one pad of the plurality of pads via a wire to transmit a channel of signals.

In some embodiments, a layout of the plurality of wires (e.g., a width of the wire, a distance between adjacent wires) in the lead part 120 may be determined based on actual requirements (e.g., a type of a subject to be detected). For example, for a relatively short device used in a rodent, the width of the wire may be in a range of 1-20 μm (e.g., 1.5 μm), and a distance between adjacent wires (e.g., a distance between center lines of adjacent wires) may be in a range of 2-60 μm (e.g., 4 μm). As another example, for a relatively long device used in a non-human primate, the width of the wire may be in a range of 1-20 μm (e.g., 3 μm), and a distance between adjacent wires may be in a range of 2-60 μm (e.g., 8 μm).

The interface part 130 may be configured to connect the device to a second device. The second device may be configured to collect, transfer, and/or process data and/or information obtained by the planar electrode array 100. In some embodiments, the second device may include a connection cable (or a connection board) connected to the interface part 130. In some embodiments, the second device may include a printed circuit board (PCB), a flexible flat cable (FFC), a flexible extender board, an integrated circuit chip (e.g., a CMOS chip), or the like, or any combination thereof.

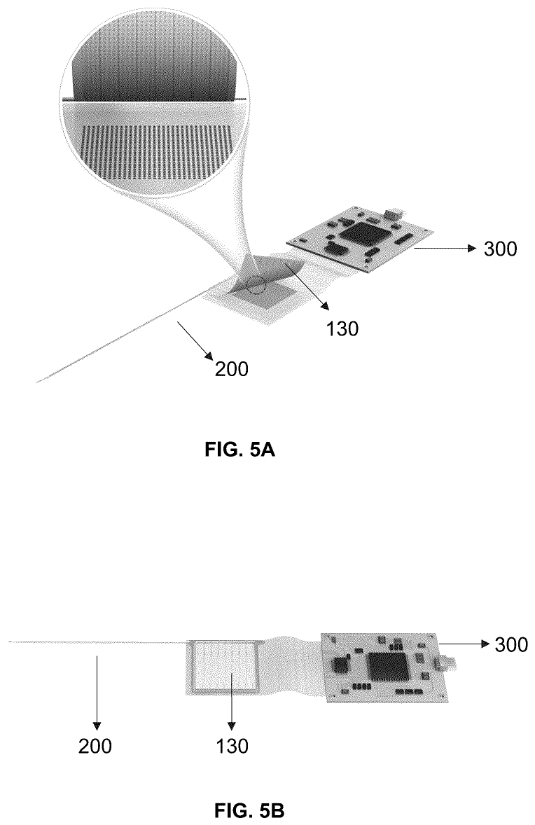

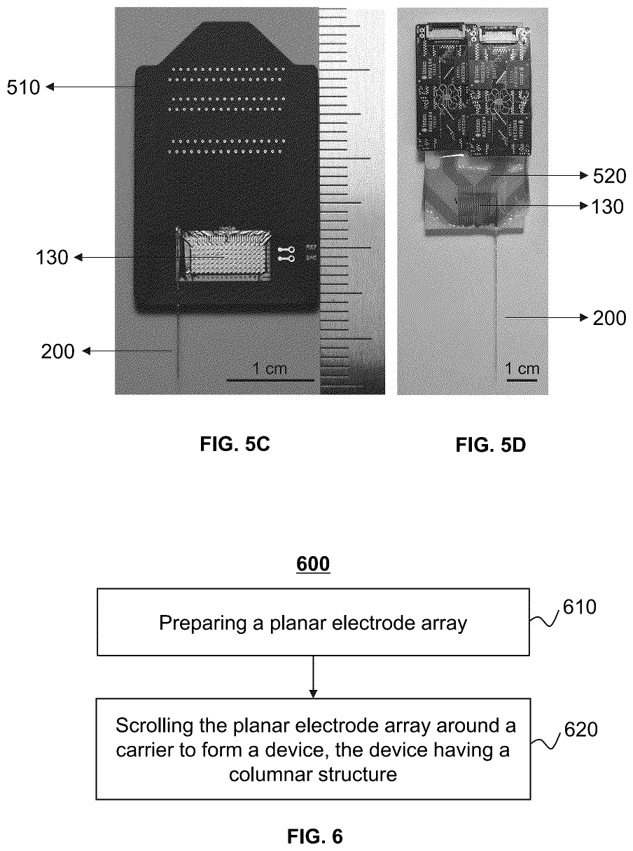

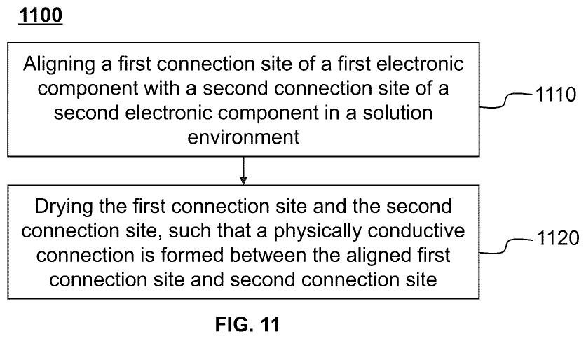

In some embodiments, the interface part 130 may be connected to the second device via any suitable connection manners. For example, the interface part 130 may be connected to the second device via a flip chip, an adhesive conducting material (e.g., a silver epoxy), a wire bonding connection, a direct bonding connection, or the like, or any combination thereof. As another example, the interface part 130 may be connected to the second device using a connection method shown in FIG. 11. More descriptions of the connection between the interface part 130 and the second device may be found elsewhere in the present disclosure (e.g., FIGS. 5A-5D, FIGS. 11-17B, and descriptions thereof).

In some embodiments, the electrode site part 110, the lead part 120, and the interface part 130 may be arranged on a same surface of the planar electrode array 100 or different surfaces of the planar electrode array 100. For example, the electrode site part 110 may be arranged on a front surface of the planar electrode array 100. The lead part 120 and the interface part 130 may be arranged on a rear surface of the planar electrode array 100.

It should be noted that the planar electrode array illustrated in FIGS. 1A-1C is merely provided for the purposes of illustration, and not intended to limit the scope of the present disclosure. The arrangement of the electrode site part 110, the lead part 120, and the interface part 130 of the planar electrode array 100 may be designed based on actual requirements (e.g., features of brain regions of the subject to be detected). For example, the electrode site part 110, the lead part 120, and the interface part 130 may be located at any positions of the planar electrode array 100.

FIGS. 2A-2C are schematic diagrams illustrating an exemplary process for preparing a device according to some embodiments of the present disclosure. FIG. 2A is a schematic diagram illustrating an exemplary connection between a planar electrode array and a carrier according to some embodiments of the present disclosure. FIG. 2B is a schematic diagram illustrating an exemplary scrolling process of a planar electrode array around a carrier according to some embodiments of the present disclosure. FIG. 2C is a schematic diagram illustrating an exemplary device according to some embodiments of the present disclosure. FIG. 2D is a picture illustrating an exemplary connection part according to some embodiments of the present disclosure.

As illustrated in FIG. 2A, the planar electrode array 100 may include a connection part 210. The connection part 210 may be configured to connect the planar electrode array 100 to a carrier 220. The carrier 220 may be configured to scroll the planar electrode array 100, improve a mechanical strength of the device, perform an optical stimulation or an electrical stimulation on a subject, and/or perform a drug delivery. For example, the carrier 220 may have a hollow structure, to perform the delivery of drugs and/or reagents on the subject. As another example, the carrier 220 may be a biomedical electrode, to perform the optical stimulation or the electrical stimulation on the subject.

In some embodiments, the carrier 220 may include a metal wire, a metal tube, a quartz wire, a quartz tube, an optical fiber, a polymer wire, a polymer tube, a ceramic wire, a ceramic tube, a rubber tube, a silicone tube, a graphene fiber, a carbon fiber, a carbon nanotube filament, a combination of wire and tube, a stereoelectroencephalography (SEEG) electrode, an intracranial pressure measuring device, or the like, or any combination thereof. The metal wire may include a tungsten wire, a Ni—Ti alloy wire, a Ni—Cr alloy wire, etc. In some embodiments, the type of the carrier 220 may be selected according to actual requirements to achieve different functions. Accordingly, by using the multifunctional integrated carrier 220, a synchronous operation of electrical measurement or stimulation and other measurements may be realized.

The carrier may have a single layer or a multi-layer structure. In some embodiments, the carrier may have a single layer structure. In some embodiments, a single-layer carrier may have a hollow structure or a solid structure. In some embodiments, the single-layer carrier may include a metal wire, a metal tube, a quartz wire, a quartz tube, an optical fiber, a polymer wire, a polymer tube, a ceramic wire, a ceramic tube, a rubber tube, a silicone tube, a graphene fiber, a carbon fiber, a carbon nanotube filament, a stereoelectroencephalography (SEEG) electrode, an intracranial pressure measuring device, or the like, or any combination thereof.

In some embodiments, a multi-layer carrier may include an outer hollow catheter and an inner guide wire. In some embodiments, the outer hollow catheter may include a metal tube, a quartz tube, a polymer tube, a ceramic tube, a rubber tube, a silicone tube, a carbon tube, or the like, or any combination thereof. The inner guide wire may include a metal wire, a quartz wire, an optical fiber, a polymer wire, a ceramic wire, a graphene fiber, a carbon fiber, a carbon nanotube filament, a stereoelectroencephalography (SEEG) electrode, an intracranial pressure measuring device, or the like, or any combination thereof. In some embodiments, the outer hollow catheter and the inner guide wire may have the same material. Alternatively, the outer hollow catheter and the inner guide wire may have different materials.

In some embodiments, a length of the carrier 220 may be in a range of several millimeters to tens of centimeters. In some embodiments, a diameter of the carrier 220 may be in a range of 5-2000 μm. For example, when the carrier 220 is the tungsten wire, a diameter of the tungsten wire may be 30 μm, 50 μm, 100 μm, etc. In some embodiments, the length and/or the diameter of the carrier 220 may be determined based on actual requirements (e.g., a type of a subject to be detected).

The connection part 210 may be configured to fix the carrier 220 on the planar electrode array 100. In some embodiments, the electrode site part 110 and the connection part 210 may be arranged on opposite sides of the planar electrode array 100. For example, the electrode site part 110 may be located on the second side (e.g., side B) of the planar electrode array 100. In some embodiments, the lead part may be of a “U” shape, the lead part may be arranged on an opposite end to that of the electrode site part and the interface part on the planar electrode array. In some embodiments, the lead part may be arranged on the two sides of the electrode site part. The carrier 220 may be fixed on the first side (e.g., side A) of the planar electrode array 100 via the connection part 210. In some embodiments, the electrode site part 110 and the connection part 210 may be arranged on opposite surfaces of the planar electrode array 100. For example, the electrode site part 110 may be arranged on the front surface of the planar electrode array 100, and the connection part 210 may be arranged on the rear surface of the planar electrode array 100.

In some embodiments, the connection part 210 may include a hole structure or a mesh structure (e.g., the connection part 210 as illustrated in FIG. 2D), such that the carrier 220 can pass through the hole structure or the mesh structure to be fixed on the connection part 210. In some embodiments, a hole of the hole structure or the mesh structure may be in a shape of a circle, a triangle, a rectangle, a square, a diamond, or other regular and/or irregular polygons.

In some embodiments, the carrier 220 may be connected to the planar electrode array 100 (e.g., the connection part 210 of the planar electrode array 100) via a bonding connection. For example, the carrier 220 may be connected to the connection part 210 via an adhesive material. The adhesive material may include a polyoxyethylene (PEO) solution, a polyethylene glycol (PEG) solution, a silk fibroin solution, a Kollicoat® (e.g., Kollicoat® MAE 100 P) solution, a biological glue, a medical glue, a sucrose solution, a gelatine, a photoresist, or the like.

In some embodiments, the carrier 220 (e.g., a metal wire, a quartz tube, an optical fiber) may be connected to the planar electrode array 100 (e.g., the connection part 210 of the planar electrode array 100) via a snap-in connection. Merely by way of example, a groove (e.g., a polymer groove) may be arranged at a side (e.g., the first side (e.g., side A) as illustrated in FIG. 2A) of the planar electrode array 100, and the carrier 220 may be fixed on the planar electrode array 100 by putting the carrier 220 into the groove. In some embodiments, the groove (e.g., the polymer groove) may be formed on the planar electrode array 100 via a micro/nano fabrication process. In some embodiments, a size of the groove may be matched with a size of the carrier 220. For example, a ratio of a size of the groove (e.g., the diameter of the groove) to a size of the carrier 220 (e.g., the diameter of the carrier 220) may be in a range of 2:1.

In some embodiments, the planar electrode array 100 may be connected to the carrier 220 after the planar electrode array 100 is released from a substrate. In some embodiments, the planar electrode array 100 may be connected to the carrier 220 before the planar electrode array 100 is released from the substrate. That is, after the planar electrode array 100 is connected to the carrier 220, the planar electrode array 100 may then be released from the substrate.

In some embodiments, the carrier 220 and the planar electrode array 100 may be configured as an integral piece. In some embodiments, the carrier 220 and the planar electrode array 100 may be fabricated by a micro/nano fabrication process. For example, the carrier 220 may be made of a photoresist (e.g., SU-8). Before the planar electrode array 100 is released from the substrate, the carrier 220 may be fabricated on a side (e.g., the first side (e.g., side A) as illustrated in FIG. 2A) of the planar electrode array 100. Then the integral piece including the planar electrode array 100 and the carrier 220 may be generated after the substrate is removed from the planar electrode array 100.

As illustrated in FIG. 2C, the target electrode array 230 may be formed by scrolling the planar electrode array 100 around the carrier 220. In some embodiments, the planar electrode array 100 may be scrolled and/or attached around the carrier 220 via a manual scroll mode. In some embodiments, the planar electrode array 100 may be scrolled and/or attached around the carrier 220 via an auto scroll mode. For example, the planar electrode array 100 may be scrolled and/or attached around the carrier 220 using a scroll device.

In some embodiments, during the scrolling process, at least a portion of the rear surface of the planar electrode array 100 (i.e., a surface of the planar electrode array 100 opposite to a surface of the planar electrode array 100 where the electrode site part 110 is located) may be coated with the adhesive material, to fix the rear surface of the planar electrode array 100 on the device 200.

In some embodiments, the planar electrode array 100 may be scrolled and/or attached around the carrier 220 in a plurality of turns. That is, a plurality of layers of planar electrode arrays 100 may be formed in a direction perpendicular to an axial direction (e.g., an axial direction M as illustrated in FIGS. 2A-2C) of the carrier 220. In some embodiments, in a last turn of the scrolling process, an edge of a second side (e.g., the second side (e.g., side B) as illustrated in FIG. 2B) of the planar electrode array 100 may be coated with the adhesive material, to fix the second side of the planar electrode array 100 on the device 200. For example, the planar electrode array 100 may include a second connection part (not shown in FIGS. 2A and 2B). The second connection part and the connection part 210 may be arranged on opposite sides of the planar electrode array 100. For example, the connection part 210 may be located on the first side (e.g., side A) of the planar electrode array 100, and the second connection part may be located on the second side (e.g., side B) of the planar electrode array 100. In some embodiments, the second connection part may include the hole structure or the mesh structure. For example, the hole may be in a shape of diamond with a side length of 50 μm. In the last turn of the scrolling process, the second connection part may be coated with the adhesive material, to fix the second side of the planar electrode array 100 on the device 200.

In some embodiments, a fourth side (e.g., side D as illustrated in FIGS. 2A and 2B) of the planar electrode array 100 may have a bevel edge structure. For example, an angle between the first side (e.g., side A as illustrated in FIG. 2A) and the fourth side (e.g., side D as illustrated in FIGS. 2A and 2B) may be in a range of 83-89 degrees. The bevel edge structure may be configured to form a conical structure (e.g., a conical structure 240 as illustrated in FIG. 2B) by scrolling the planar electrode array 100 around the carrier 220. The conical structure may be at a tip of the device 200. The conical structure may facilitate the implantation of the device 200 into the subject (e.g., neural tissue of a human or an animal).

In some embodiments, the fourth side (e.g., side D as illustrated in FIGS. 2A and 2B) of the planar electrode array 100 may have a horizontal edge structure. The horizontal edge structure may be configured to form a column structure (not shown in FIGS. 2A-2C) by scrolling the planar electrode array 100 around the carrier 220.

In some embodiments, after the planar electrode array 100 is scrolled and/or attached around the carrier 220, the electrode site part 110 may be exposed on a surface (e.g., an external surface) of the device 200, and the lead part 120 (not shown in FIG. 2C) may be embedded in an internal part of the device 200 or exposed on a surface of the device 200, as illustrated in FIG. 2C. In some embodiments, the device 200 formed by scrolling the planar electrode array 100 around the carrier 220 may be like a “flag.” As used herein, an “external surface (or external part)” may refer to a surface (or part) that is exposed to air or contacts surfaces of another component and an “internal surface (or internal part)” may refer to a surface (or part) that is not exposed to air or invisible from the outside.

In some embodiments, after the device 200 is formed, the carrier 220 may be fully or partly left with the device 200 or removed from the device 200. In some embodiments, by scrolling the planar electrode array 100 around the carrier 220 to form the device 200, ever after the carrier 220 is removed from the device 200, the mechanical strength of the device 200 may be relatively high.

In some embodiments, a conducting layer of the target electrode array 230 may be made of gold, platinum, copper, aluminum, silver, titanium, chromium, nickel, tantalum, palladium, molybdenum, a carbon nanotube (CNT), a graphene, a carbon material, iridium oxide, titanium nitride, a conducting polymer, indium tin oxide, tantalum oxide, a liquid metal, or the like, or any combination thereof. In some embodiments, a length of the target electrode array along a longitudinal axis of the device 200 may be in a range of several millimeters to tens of centimeters.

It should be noted that the planar electrode array illustrated in FIGS. 2A-2D is merely provided for the purposes of illustration, and not intended to limit the scope of the present disclosure. The arrangement of the carrier 220 and the connection part 210 may be designed based on actual requirements.

FIG. 3 is a micrograph picture illustrating an exemplary distribution of a plurality of electrode sites in a target electrode array according to some embodiments of the present disclosure.

As illustrated in FIG. 3, after the planar electrode array 100 is scrolled and/or attached around the carrier 220, a plurality of electrode sites 111 may be distributed circumferentially around the surface of the device and/or axially along the surface of the device to form the target electrode array 230. In some embodiments, the plurality of electrode sites 111 may be distributed on the entire surface of the device or a part of the surface of the device.

In some embodiments, the plurality of electrode sites 111 may be arranged in an array. The array may include a plurality of columns of electrode sites 111 and a plurality of rows of electrode sites 111. Different columns or rows may include the same number (or count) or different numbers (or counts) of electrode sites 111. As illustrated in FIG. 3, the plurality of electrode sites 111 may be arranged in a staggered array.

In some embodiments, a count of the plurality of electrode sites 111 of the target electrode array 230 may be in a range of 1-50000. In some embodiments, a size of the electrode site 111 of the target electrode array 230 may be in a range of 1-3000 μm. In some embodiments, the count of the plurality of electrode sites 111 and/or the size of the electrode site 111 may be determined based on actual requirements.

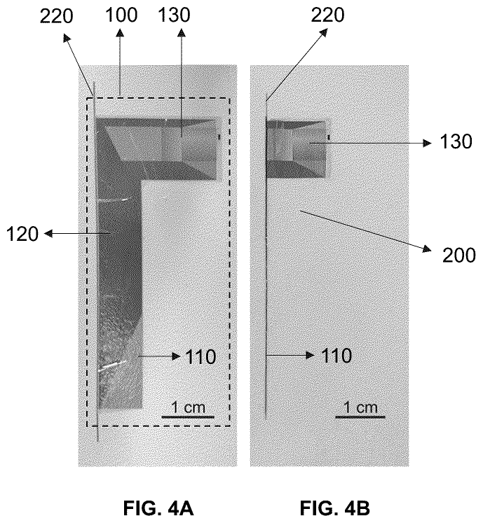

FIG. 4A is a picture illustrating an exemplary connection between a planar electrode array and a carrier according to some embodiments of the present disclosure. FIG. 4B is a picture of a device according to some embodiments of the present disclosure.

As illustrated in FIG. 4A, the planar electrode array 100 may include the electrode site part 110, the lead part 120, and the interface part 130. The carrier 220 may be fixed on a side of the planar electrode array 100 before the planar electrode array 100 is scrolled and/or attached around the carrier 220.

As illustrated in FIG. 4B, the planar electrode array 100 may be scrolled and/or attached around the carrier 220 to form the device 200. After the planar electrode array 100 is scrolled and/or attached around the carrier 220, the electrode site part 110 may be exposed on an external surface of the device 200, and the lead part 120 may be embedded in an internal part of the device 200 or exposed on a surface of the device 200. The interface part 130 may connect the device 200 to a second device as described elsewhere in the present disclosure. For example, the interface part 130 may connect to a connection cable (or a connection board) of the second device.

In some embodiments, a length of the device 200 along a longitudinal axis of the carrier 220 may be in a range of several millimeters to tens of centimeters. For example, a length of a part of the device 200 implanted in tissue of a rodent may be 1 cm. That is, for the device 200 implanted in the tissue of the rodent, a length of a target electrode array along a longitudinal axis of the carrier of the device 200 may be 1 cm. As another example, a length of a part of the device 200 implanted in tissue of a non-human primate may be equal to or greater than 5 cm. That is, for the device 200 implanted in the tissue of the non-human primate, a length of a target electrode array along the longitudinal axis of the carrier of the device 200 may be equal to or greater than 5 cm. In some embodiments, a diameter of the device 200 may be in a range of 10-3000 μm.

FIGS. 5A and 5B are schematic diagrams illustrating an exemplary connection between a device and a second device according to some embodiments of the present disclosure. FIGS. 5C and 5D are pictures illustrating an exemplary connection between a device and a second device according to some embodiments of the present disclosure. As illustrated in FIGS. 5A and 5B, the device 200 may be connected to a second device 300 via the interface part 130. The second device 300 may be configured to collect, transfer, and/or process data and/or information obtained by the device 200, and/or transfer stimulation pulses to device 200. In some embodiments, the second device 300 may include a PCB (e.g., a PCB 510 as illustrated in FIG. 5C), an FFC, a flexible extender board (e.g., a flexible extender board 520 as illustrated in FIG. 5D), etc. In some embodiments, the interface part 130 may be connected to the second device 300 via any suitable connection manner. For example, the interface part 130 may be connected to the second device 300 via a flip chip, an adhesive conducting material (e.g., a silver epoxy), a wire bonding connection, a direct bonding connection (e.g., a connection as illustrated in FIGS. 5C and 5D), or the like, or any combination thereof. As another example, the device (e.g., the interface part 130 of the device) may be connected to the second device 300 using a connection method shown in FIG. 11. In some embodiments, the device may include a first connection site (e.g., the interface part 130), the second device may include a second connection site. The first connection site of the device may be aligned with the second connection site of the second device in a solution environment. The first connection site and the second connection site may be dried such that a physically electrically conductive connection is formed between the aligned first connection site and second connection site. More descriptions of the exemplary connection method can be found elsewhere in the present disclosure (e.g., FIGS. 11, 12, 13, 14A, 14B, 16, 17A, 17B and descriptions thereof).

FIG. 6 is a flowchart illustrating an exemplary process for preparing a device according to some embodiments of the present disclosure. Operations of the illustrated process presented below are intended to be illustrative. In some embodiments, a process 600 may be accomplished with one or more additional operations not described and/or without one or more of the operations discussed. Additionally, the order of the operations of process 600 illustrated in FIG. 6 and described below is not intended to be limiting.

In 610, a planar electrode array (e.g., the planar electrode array 100 as illustrated in FIGS. 1A-1C, 2A, 2B, and 4A) may be prepared.

In some embodiments, the planar electrode array may be fabricated by a micro/nano fabrication process (e.g., a micro-electro-mechanical system (MEMS) technology, a CMOS technology). In some embodiments, the planar electrode array may be fabricated based on a thin film deposition technology, a physical vapor deposition (PVD) technology, a photolithography technology, an etching technology (e.g., a reactive ion etching technology), or the like, or any combination thereof.

In some embodiments, the planar electrode array may include a substrate, a conductive layer, and an encapsulated layer. In some embodiments, the substrate and/or the encapsulated layer of the planar electrode array may be made of a flexible material. For example, the substrate and/or the encapsulated layer of the planar electrode array may be made of polyimide (PI), Parylene-C, polydimethylsiloxane (PDMS), polystyrene-block-poly(ethylene-ran-butylene)-block-polystyrene (SEBS), SU-8 photoresist, polyethylene terephthalate (PET), polyurethane (PU), Ecoflex, poly(styrene-butadiene-styrene) (SBS), Teflon, or the like, or any combination thereof.

In some embodiments, a nickel-deposited silicon wafer may be prepared by depositing nickel on a silicon wafer. The nickel may be determined as a sacrificial layer of the planar electrode array. For example, the nickel may be deposited on the silicon wafer via a magnetron sputtering technology. In some embodiments, a thickness of the sacrificial layer may be in a range of 50-200 nm. The substrate of the planar electrode array may be prepared by spin coating, heating, and curing polyamic acid, PDMS, or SU-8 photoresist, or by depositing Parylene-C, on the nickel-deposited silicon wafer. In some embodiments, a thickness of the substrate may be in a range of 50-50000 nm. For example, the thickness of the substrate may be 500 nm.

A conductive layer of the planar electrode array may be prepared by depositing and patterning at least one of a metal (e.g., chromium (Cr)/gold (Au), platinum), a carbon nanotube, a graphene, a carbon material, iridium oxide, titanium nitride, indium tin oxide, tantalum oxide, a conductive polymer on the substrate of the planar electrode array. The conductive layer may be configured to record physiological signals and/or achieve an electrical connection to an external device. For example, a patterned metal layer may be prepared by depositing Cr/Au on the substrate via a photolithography technology and a magnetron sputtering technology.

An encapsulated layer of the planar electrode array may be prepared by spin coating, heating, and curing at least one of polyamic acid, PDMS, SEBS, SU-8 photoresist, or by depositing Parylene-C, on the conductive layer of the planar electrode array. For example, the encapsulated layer may be prepared on the patterned metal layer. In some embodiments, a thickness of the encapsulated layer may be in a range of 50-50000 nm. For example, the thickness of the encapsulated layer of the planar electrode array may be 500 nm.

A mask may be prepared by depositing and patterning aluminium (Al) on the encapsulated layer of the planar electrode array to obtain a masked encapsulated layer. For example, the mask may be prepared by depositing and patterning Al on the encapsulated layer via a photolithography and magnetron sputtering technology.

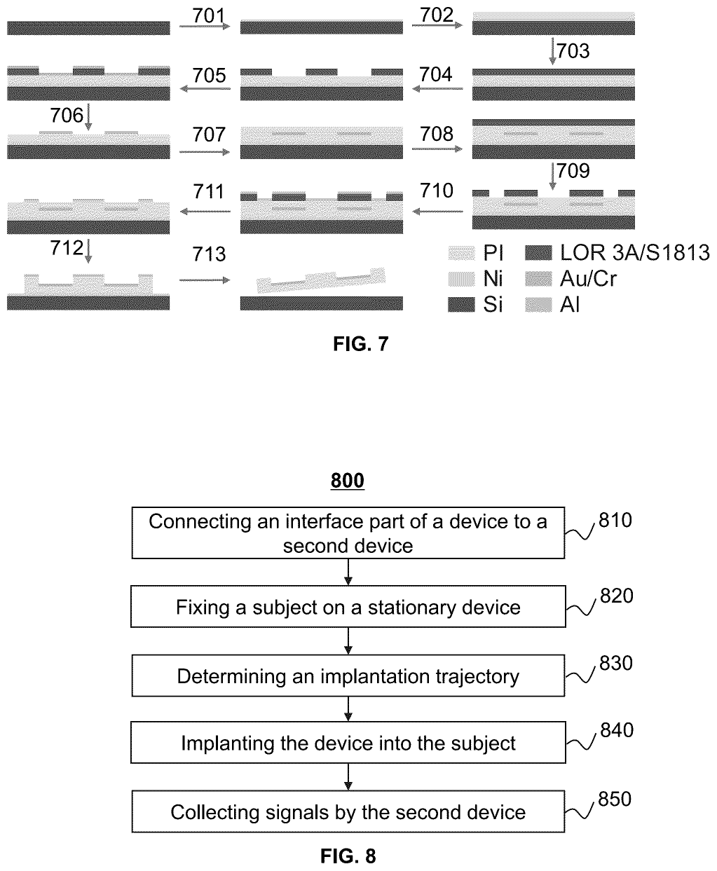

A patterned electrode array may be prepared by processing the masked encapsulated layer using reactive ion etching. In some embodiments, electrode sites and an interface part may be exposed on the patterned electrode array by processing the masked encapsulated layer. The planar electrode array may be obtained by etching the mask and the sacrificial layer from the silicon wafer using ferric chloride solution. For example, the planar electrode array may be released by etching the mask and the sacrificial layer from the silicon wafer using ferric chloride solution. The planar electrode array may be cleaned repeatedly using deionized water. More descriptions for preparing the planar electrode array may be found elsewhere in the present disclosure (e.g., FIG. 7, and descriptions thereof).

In some embodiments, the operation for preparing the encapsulated layer of the planar electrode array, the operation for preparing the masked encapsulated layer, and the operation for processing the masked encapsulated layer may be omitted. For example, after the conductive layer of the planar electrode array is prepared, the planar electrode array may be released by etching the sacrificial layer from the silicon wafer using the ferric chloride solution.

In 620, the planar electrode array may be scrolled and/or attached around a carrier (e.g., the carrier 220 as illustrated in FIGS. 2A-2C, 4A, and 4B) to from a device (e.g., the device 200 as illustrated in FIGS. 2C, 4B, 5A, and 5B). The device may have a columnar structure.

In some embodiments, after the planar electrode array is released, the carrier may be fixed on a first side (e.g., side A as illustrated in FIGS. 1A, 1B, and 2A) of the planar electrode array. The planar electrode array may be scrolled and/or attached around the carrier. In a last turn of the scrolling, an edge of a second side (e.g., side B as illustrated in FIGS. 1A, 1B, 2A, and 2B) of the planar electrode array may be coated with an adhesive material, to fix the second side of the planar electrode array onto the device. More descriptions for scrolling the planar electrode array around the carrier to form a device may be found elsewhere in the present disclosure (e.g., FIGS. 2A-2C and descriptions thereof).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 7 is a schematic diagram illustrating an exemplary process for preparing a planar electrode array according to some embodiments of the present disclosure. Operations of the illustrated process presented below are intended to be illustrative. In some embodiments, a process 700 may be accomplished with one or more additional operations not described and/or without one or more of the operations discussed. Additionally, the order of the operations of process 700 illustrated in FIG. 7 and described below is not intended to be limiting.

In operation 701, a nickel-deposited silicon wafer may be prepared by depositing nickel on a silicon wafer via a magnetron sputtering technology. In operation 702, a substrate may be prepared by spin coating, heating, and curing polyamic acid on the nickel-deposited silicon wafer. In operation 703, LOR 3A lift-off resist and S1813 photoresist may be spin-coated on the substrate. In operation 704, the coated photoresist may be patterned to obtain a patterned photoresist using photolithorgraphy. In operation 705, Cr/Au may be deposited on the substrate and the patterned photoresist via a magnetron sputtering technology. In operation 706, a conductive layer may be obtained by removing the patterned photoresist via a metal lift-off technology.

In operation 707, an encapsulated layer may be prepared by spin coating, heating, and curing the polyamic acid on the conductive layer. In operation 708, LOR 3A lift-off resist and S1813 photoresist may be spin-coated on the encapsulated layer. In operation 709, the coated photoresist may be patterned to obtain a patterned photoresist using photolithorgraphy. In operation 710, an Al mask may be prepared by depositing and patterning Al on the encapsulated layer and the patterned photoresist to obtain a masked encapsulated layer. In operation 711, the patterned photoresist may be removed via the metal lift-off technology. In operation 712, a patterned electrode array may be obtained by processing the masked encapsulated layer using reactive ion etching. In operation 713, a planar electrode array may be released by etching the Al mask and the sacrificial layer from the silicon wafer using ferric chloride solution. The planar electrode array may be a three-layer structure including the substrate, the conductive layer, and the encapsulated layer.

In some embodiments, operations 707-712 may be omitted. In this situation, in operation 713, the planar electrode array may be released by etching the sacrificial layer from the silicon wafer using the ferric chloride solution.

FIG. 8 is a flowchart illustrating an exemplary process for implanting a device into a subject according to some embodiments of the present disclosure. Operations of the illustrated process presented below are intended to be illustrative. In some embodiments, a process 800 may be accomplished with one or more additional operations not described and/or without one or more of the operations discussed. Additionally, the order of the operations of process 800 illustrated in FIG. 8 and described below is not intended to be limiting.

In 810, an interface part (e.g., the interface part 130 as illustrated in FIGS. 1A, 1B, 2A-2C, 4A, 4B, 5A, 5B, 5C and 5D) of a device (e.g., the device 200 as illustrated in FIGS. 2C, 4B, 5A, 5B, 5C and 5D) may be connected to a second device (e.g., the second device 300 as illustrated in FIGS. 5A and 5B).

More descriptions for connecting the interface part to the second device may be found elsewhere in the present disclosure (e.g., FIGS. 1A, 1B, 5A, 5B, and descriptions thereof).

In 820, a subject may be fixed on a stationary device.

The subject may be a patient, an animal (e.g., a rodent, a non-human primate), or the like. The stationary device may be configured to fix a position and/or a posture of the subject to keep the subject in a rest state, so as to facilitate an implantation of the device into the subject. For example, the stationary device may include a stereotactic apparatus.

In 830, an implantation trajectory may be determined.

The implantation trajectory may be configured to guide the device to be implanted into the subject or a portion (e.g., brain tissue) thereof. In some embodiments, the implantation trajectory may be determined based on actual requirements (e.g., a type of the subject, a type of detected parameter of the subject). In some embodiments, the implantation trajectory may be determined based on a surgical navigation system.

In 840, the device may be implanted into the subject.

In some embodiments, after a craniotomy is performed on the subject, and dura mater of the subject is removed, the device may be implanted into the subject. For example, the device may be fixed on a holding device (e.g., an electrode holder), and the device may be implanted into the subject by controlling a movement of the holding device.

In 850, signals may be collected by the second device.

For example, the device may detect neural signals in a deep brain region of the subject. The second device may obtain the detected neural signals from the device for further process and storage.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

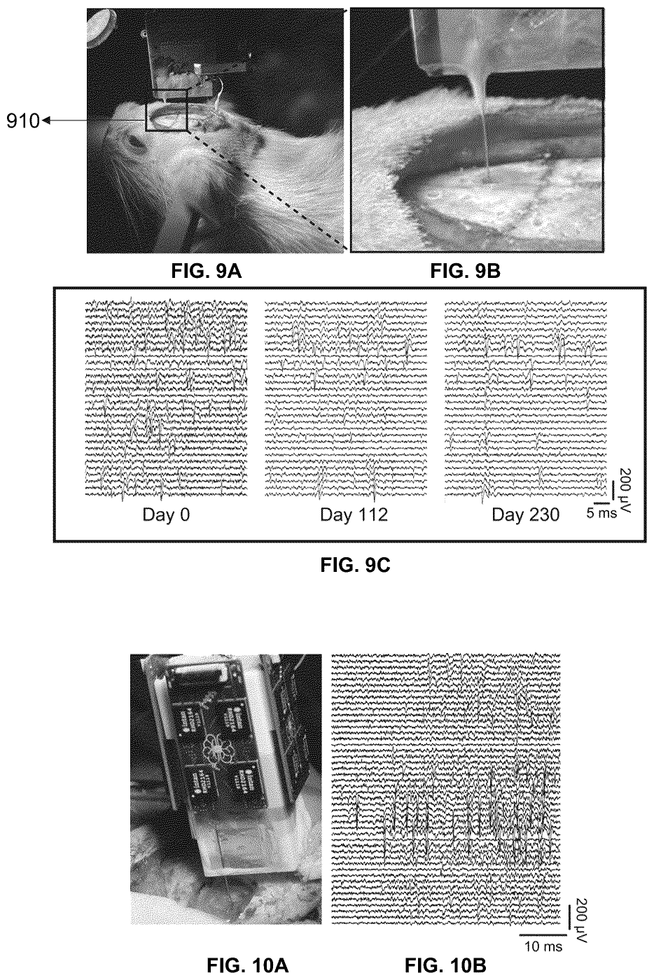

FIG. 9A is a picture illustrating an exemplary process for implanting a device into the brain of a rat according to some embodiments of the present disclosure. FIG. 9B is an enlarged view of a region 910 shown in FIG. 9A according to some embodiments of the present disclosure. FIG. 9C is a picture illustrating neural signals recorded in a rat according to some embodiments of the present disclosure.

In some embodiments, a device (e.g., the device 200 as illustrated in FIGS. 2C, 4B, 5A, 5B and 5C) may be implanted into a rodent. A craniotomy may be performed by drilling a hole on the skull using a surgical drill. The dura mater of the rat may be removed. The device (e.g., the device 200 as illustrated in FIGS. 2C, 4B, 5A, 5B and 5D) may be implanted into the brain tissue of the rat. After the device is implanted in the brain tissue of the rat, the craniotomy may be sealed. The device may be fixed via a dental cement, or the like. The device may be configured to perform a long-term monitoring of a neural activity including action potentials and field potentials (e.g., neural signals as illustrated in FIG. 9C) of the rat. For example, the device may record neuron action potentials and field potentials with a high signal-to-noise ratio (SNR) in 230 days after the device is implanted into the brain of the rat, as illustrated in FIG. 9C.

FIG. 10A is a picture illustrating an exemplary process for implanting a device into the brain of a rhesus monkey according to some embodiments of the present disclosure. FIG. 10B is a picture illustrating neural signals recorded in rhesus monkey according to some embodiments of the present disclosure.

In some embodiments, the device (e.g., the device 200 as illustrated in FIGS. 2C, 4B, 5A, 5B and 5D) may be implanted into a non-human primate. For example, a rhesus monkey may be fixed on a stationary device (e.g., a stereotactic apparatus) and the skull of the rhesus monkey may be exposed. A craniotomy point and an implantation trajectory may be determined based on a surgical navigation system. The dura mater of the rhesus monkey may be removed. The device (e.g., an electronic probe with 1024 channels) may be implanted into the brain tissue of the rhesus monkey, and the device may reach a deep brain region of the rhesus monkey. As illustrated in FIG. 10A, after the device is implanted in the brain tissue of the rhesus monkey, the bone window may be sealed. The device may be fixed via a dental cement, or the like. The scalp of the rhesus monkey may be sutured. The device may be configured for intraoperative recording and/or long-term chronic recording of neural activity, including neural action potentials, and field potentials (e.g., neural signals as illustrated in FIG. 10B) of the rhesus monkey.

In some embodiments, a carrier of the device may have a multi-layer structure. The multi-layer carrier may include a combination of a hollow catheter and a guide wire. The carrier may be fully or partly removed from the device before the process of implant surgery, in the process of implant surgery, or after the surgery. In some embodiments, an optical stimulation, an electrical stimulation, and/or a delivery of drugs and/or reagents may be performed on the subject through the guide wire or hollow catheter, in the process of implant surgery, or after the surgery. Additionally or alternatively, one or more other functional devices (e.g., an optical fiber, a SEEG electrode, a drug delivery cannula, an intracranial pressure measuring device, etc.) may be inserted into the hollow catheter to implement corresponding function(s) in the process of implant surgery, or after the surgery. The configuration of the multi-layer structure can improve the functionality of the device.

In some embodiments, multiple devices may be implanted into the rhesus monkey via multiple implantation trajectories. For example, a first device may be implanted into the rhesus monkey via a first implantation trajectory. The first implantation trajectory may pass through the primary visual cortex and the hippocampus of the rhesus monkey. A second device may be implanted into the rhesus monkey via a second implantation trajectory. The second implantation trajectory may pass through the cortex and the caudate nucleus of the rhesus monkey. A third device may be implanted into the rhesus monkey via a third implantation trajectory. The third implantation trajectory may pass through the cortex, the caudate nucleus, and the hippocampus of the rhesus monkey.

According to some embodiments of the present disclosure, the device may be applied to the brain, the spinal cord, peripheral nerves, an organ, or the like, of the subject for intraoperative recording and/or long-term chronic monitoring of the neural activity, and other biological signals of the subject. In addition, the device may be used in electrophysiological signal recording and stimulation, neurochemical recording, biochemistry and pressure sensing.

At present, methods for connecting electronic components may include flip chip connection, silver epoxy connection, and wire bonding. The flip chip is a kind of integrated circuit packaging technology, which includes hot pressing flip chip, reflow soldering, etc. The flip chip method directly deposits a welding point (e.g., tin or other low melting point metal(s) or alloy(s)) on each target site, and then another chip is flipped. After the target site(s) and the chip are aligned, the welding point(s) can connect the target site(s) and the chip under heating and/or pressurization (or through high temperature which makes solder of the welding point(s) melt and then solidify). However, a relatively high temperature may cause damage to some electronic components (such as flexible electronic components). The silver epoxy used in silver epoxy connection is obtained by mixing the silver particles with resin, the silver epoxy connection and the flip chip connection are basically the same. Silver epoxy can be coated on the surface of an external electronic component (such as a printed circuit board) to form one or more connection points, and then the chip need to be connected may be flipped on it. The silver epoxy may solidify under heating (such as heating to 85° C. or so) and pressurization (such as pressurized to 5-10 N), to form the connection. The wire bonding is another integrated circuit packaging technology, which uses a metal micro-wire to connect two metal pads. The wire bonding may include a three-step process: in a first step, ultrasonic waves may be used to melt the wire into a ball and the ball may be pressed down onto a substrate to form a solder joint; in a second step, when the wire is connected to a first solder joint, the wire is stretched and extended to a second solder joint; and in a third step, the wire is pressed directly down onto the substrate and is broken to form the connection. The wire bonding method may be unsuitable for the connection of non-fixed devices because of the thin metal micro-wire.

As can be seen from the above, traditional soldering techniques for connecting electronic components impose relatively strict requirements on external conditions such as temperature, pressure, and other requirements as well as the type of device, and are relatively complex to operate, which improve the difficulty in separation after connection and re-connection after separation. Some embodiments of the present disclosure provide a reversible method for connecting electronic components which can be realized under conventional conditions. The method for connecting electronic components provided in the embodiments of the present disclosure is described in detail below in conjunction with the accompanying drawings.

FIG. 11 is a flowchart illustrating an exemplary process for connecting electronic components according to some embodiments of the present disclosure. Referring to the process 100 shown in FIG. 11, the process for connecting electronic components provided in some embodiments of the present disclosure may include one or more of the following operations.

In 1110, one or more first connection sites of a first electronic component may be aligned with one or more second connection sites of a second electronic component in a solution environment. For example, a first connection site may be aligned with a second connection site in the solution environment.

The first electronic component may be or include a device (e.g., the device 200). In some embodiments, the first electronic component may include the first connection site(s). A first connection site may refer to a connection structure such as an electrode, a connection terminal, an input/output pad, or a connection channel of the first electronic component for making a connection with another electronic component (such as a second electronic component). For example, the first connection site(s) may be or include the input/output pad(s) of electrode site(s) 111 shown in FIGS. 1A, 2A, and 3. The second electronic component may be or include another device (e.g., a second device). The second device (or the second electronic component) may include a printed circuit board (PCB), a flexible flat cable (FFC), a flexible extender board, or a chip, or the like. More descriptions about the second device may be found elsewhere in the present disclosure. It should be noted that the device and the second device are merely provided for illustration purposes, the connection method shown in FIG. 11 can be applied to any suitable electronic components which are not limited to the device and the second device. The second electronic component may include one or more second connection sites. Similarly, a second connection site may refer to a connection structure such as the input/output pad(s) of an electrode, a connection terminal, or a connection channel of the second electronic component for making a connection with another electronic component (e.g., the first electronic component). In some embodiments, the first electronic component may include two or more first connection sites. In some embodiments, similarly, the second electronic component may include two or more second connection sites.

In some embodiments, the first electronic component may include or be a flexible electronic component (e.g., a flexible display, a chemical and biological sensor chip (e.g., an electrophysiological electrode array), a flexible wearable electronic component, etc.). The flexible electronic component may be stretched and bent without damaging its own electronic properties. In some embodiments, the flexible electronic component may include an implanted electrode to be implanted into a target subject (e.g., a human body, an animal, etc.). The implanted electrode may be used to be implanted into a designated part of the target subject (e.g., a surface of the cerebral cortex or a deep region in the brain of the target subject) to stimulate the target subject or to monitor physiological parameters of the target subject.

In some embodiments, the first connection site(s) of the first electronic component may include a first electrically conductive structure (also referred to as a first conductive structure), which may enable the first electronic component to be electrically conductive to another electronic component when being connected to the electronic component. In some embodiments, the first electrically conductive structure may be made of a metal-conductive material and/or a non-metal-conductive material. Exemplary metal-conductive materials may include chromium, gold, copper, silver, platinum, aluminum, alloy (e.g., copper alloy, aluminum alloy, platinum/iridium, etc.), etc. Exemplary non-metal-conductive materials may include carbon-based materials, graphene, etc.

In some embodiments, the second electronic component may include any one of a printed circuit board (PCB), a flexible flat cable (FFC), a flexible circuit board, a micromachined chip, a complementary metal oxide semiconductor (CMOS) chip, an integrated circuit chip, and a CMOS metal microelectrode array. Similar to the first electronic component, the second connection site(s) of the second electronic component may include a second conductive structure, which may be made of the same, similar, or different conductive material as the first electrically conductive structure, and may be used to connect with the first electrically conductive structure of the first electronic component and cause the first electronic component and the second electronic component to be electrically conducting.

In some embodiments, the first connection site(s) of the first electronic component may include an ultra-thin flexible connection unit (e.g., a thickness of the first connection site(s) may be of micrometer or sub-micrometer level). By using the ultra-thin flexible connection unit, it is possible to enable the first connection site(s) of the first electronic component to realize the connection with the second electronic component without heating or pressurization. For more information about the connection principle of the first electronic component and the second electronic component, please refer to other descriptions in the present disclosure, which will not be repeated herein.

In some embodiments of the present disclosure, the thickness of the first connection site(s) may be between 50 nm to 5 μm. Exemplarily, in some embodiments, the thickness of the first connection site(s) may be between 80 nm to 1.5 μm; in some embodiments, the thickness of the first connection site(s) may be between 100 nm to 1 μm.

In some embodiments, the first connection site(s) and/or the second connection site(s) may have a mesh structure.

In some embodiments, the first connection site(s) of the first electronic component may be aligned with the second connection site(s) of the second electronic component in a solution environment. The solution environment may refer to an environment created by dripping a solution at the connection area between the first connection site(s) and the second connection site(s). In some embodiments, the solution may include water (e.g., deionized water), ethanol, isopropanol, etc. It can be understood that the above solutions are provided for illustration only, and in embodiments of the present disclosure, the solution may include, but is not limited to, the types descripted above. In some embodiments, in order to facilitate drying of the solution in a subsequent process, a volatile solution (e.g., anhydrous ethanol, 75% ethanol, etc.) may be used in 1110.

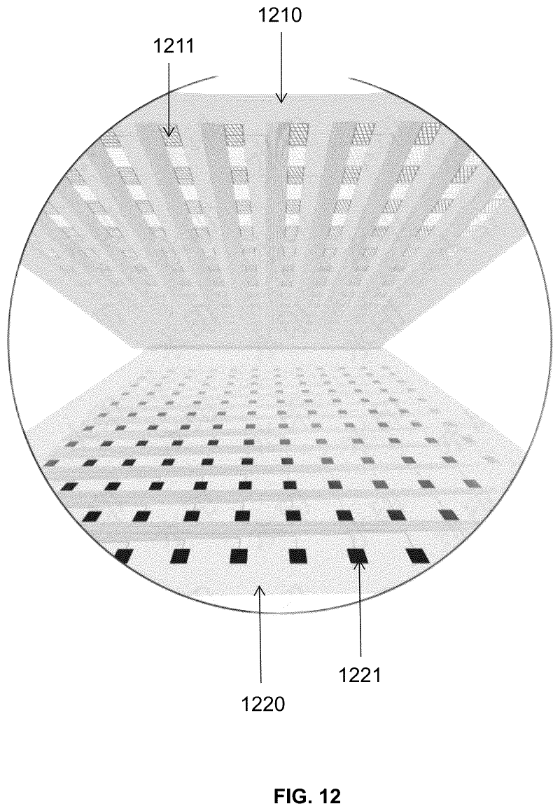

FIG. 12 is a schematic diagram illustrating an exemplary process for aligning electronic components to be connected according to some embodiments of the present disclosure.

Referring to FIG. 12, in some embodiments, the first electronic component 1210 may be spread out using tweezer(s) (e.g., pointed tweezer(s)) in the solution environment, and then, one or more first connection sites 1211 thereon may be aligned with one or more second connection sites 1221 on the second electronic component 1220, respectively. In some embodiments, the alignment operation may be performed under a stereoscope.

It should be noted that the one or more first connection sites 1211 on the first electronic component 1210 and the one or more second connection sites 1221 on the second electronic component 1220 may have the same or similar arrangement or spacings. If the first electronic component 1210 and/or the second electronic component 1220 have a plurality of connection sites, the alignment therebetween can be realized by aligning only a portion of the plurality of connection sites. Each aligned first connection site 1211 may overlap or partially overlap with its corresponding second connection site 1221.

Continuing to refer to the process 1100 shown in FIG. 11, the process for connecting electronic components provided in some embodiments of the present disclosure may further include one or more of the following operations.

In 1120, the first connection site(s) and the second connection site(s) may be dried, such that a physically electrically conductive connection is formed between the aligned first connection site and second connection site.

In some embodiments, after aligning the first connection site(s) of the first electronic component and the second connection site(s) of the second electronic component in 1110, the first connection site(s) and the second connection site(s) may be dried, so that after removing the solution, a physically electrically conductive connection is formed between the aligned first connection site(s) and second connection site(s). In the present disclosure, the term “physically electrically conductive connection” refers to a connection relationship formed by utilizing physical characteristics (e.g., shape, structure, etc.) of the first connection site(s) and the second connection site(s), which allows a certain connection force to be generated between the first connection site(s) and the second connection site(s) and makes the first connection site(s) and the second connection site(s) be electrically conducting.

It can be understood that in the embodiment of the present disclosure, since the thickness of the first connection site(s) of the first electronic component is relatively thin and the first connection site(s) have a flexible structure, when drying the solution, the first connection site(s) may be bent to the second connection site(s) under the action of a surface tension of the solution and/or an adsorption force of the solution and the first connection site(s), so as to enable the formation of a physically electrically conductive connection between the first connection site(s) and the second connection site(s).

In some embodiments, the physically electrically conductive connection formed between the first connection site(s) and the second connection site(s) may enable electrical conduction between the first connection site(s) and the second connection site(s). In some embodiments, a contact resistance between the first connection site(s) and the second connection site(s) is less than 10 MΩ. The contact resistance may be referred to as an additional resistance presented between the first connection site(s) and the second connection site(s). The contact resistance may be related to the size, structure, and material of the electronic component(s) thereof. In some embodiments, the contact resistance may be used to characterize the tightness of the connection between the first connection site(s) and the second connection site(s). Specifically, in some embodiments, according to experimental results, the contact resistance between the first connection site(s) and the second connection site(s) may be generally between 100Ω and 100 kΩ.

It should be noted that in the present disclosure, the first connection site(s) and the second connection site(s) may be dried using any method that does not affect the connection relationship between the first connection site(s) and the second connection site(s). Exemplarily, in some embodiments, the method for drying the first connection site(s) and the second connection site(s) may include squeezing, blow-drying, oven drying, drying naturally in the air, or removing the solution using absorbent paper. It should be noted that in the present disclosure, the method for drying the first connection site(s) and the second connection site(s) may be any method that does not affect the connection relationship between the first connection site affect and the second connection site affect.

It should be noted that the above descriptions of the process 1100 is intended to be exemplary and illustrative only and does not limit the scope of application of the present disclosure. For those skilled in the art, various amendments and changes to the process 1100 may be made under the guidance of the present disclosure, such amendments and changes remain within the scope of the present disclosure.

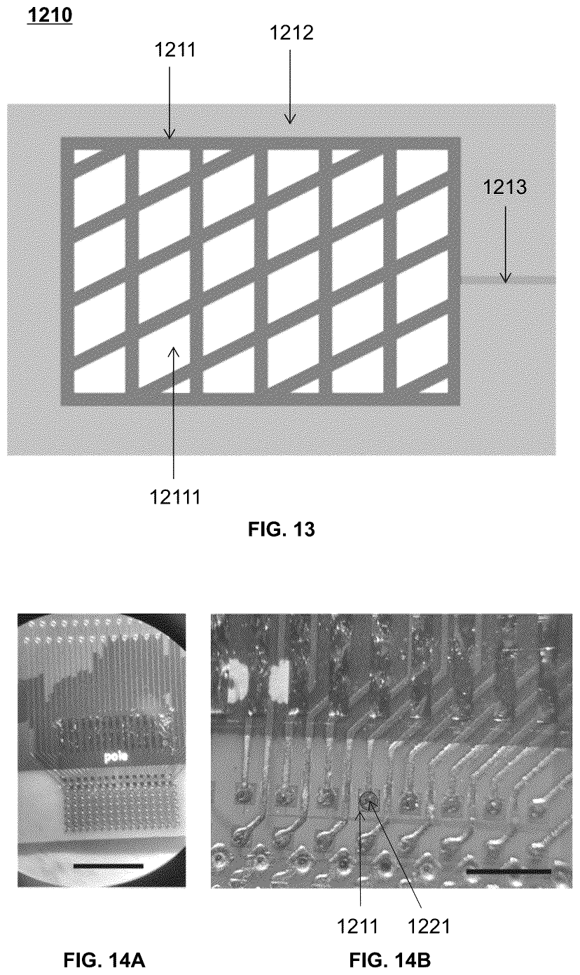

FIG. 13 is a schematic diagram illustrating an exemplary structure of a first connection site of a first electronic component according to some embodiments of the present disclosure.

Referring to FIG. 13, in some embodiments, a first connection site 1211 of the first electronic component 1210 may have a mesh conductive structure, and the mesh conductive structure may include a skeletonized portion 12111. By using the mesh conductive structure, a micro chamber may be formed in the skeletonized portion 12111, which may hold a certain amount of solution in the solution environment. After removing the solution in the micro chamber through drying, a negative pressure environment may be formed between the first connection site 1211 of the first electronic component and the second connection site of the second electronic component, so that the first connection site 1211 of the first electronic component and the second connection site of the second electronic component are adsorbed with each other under the action of a liquid surface tension, a negative pressure adsorption force, and a physical adsorption force (also known as van der Waals adsorption, caused by intermolecular force(s)), thereby strengthening to a certain extent the connection force between the first connection site 1211 and the second connection site.

It should be noted that the structure shown in FIG. 13 is only an exemplary illustration. In some embodiments, the shape of the skeletonized portion 12111 of the mesh conductive structure may be, but is not limited to, rhombus. In some embodiments, the shape of the skeletonized portion 12111 of the mesh conductive structure may be a regular shape such as a rectangle, a triangle, a trapezoid, or other irregular shape.

In some embodiments, an area of the first connection site in the first electronic component may be larger than an area of the second connection site in the second electronic component. When the first connection site and the second connection site are aligned, the second connection site may be covered by the first connection site, thereby maximizing a fitting area between the first connection site and the second connection site, and maximizing a connection quality (e.g., a connection stability) between the first connection site and the second connection site. It should be noted that the above relationship between the area of the first connection site and the area of the second connection site is only for exemplary illustration, and in some embodiments, the area of the first connection site may be smaller than or equal to the area of the second connection site. In some embodiments, in order to ensure a connection force after the first connection site is connected to the second connection site, a connection area (i.e., an area of the overlapping area thereof) of the first connection site and the second connection site may be made greater than or equal to 0.1 μm². Exemplarily, in some embodiments, the connection area of the first connection site and the second connection site may be in a range from 1 μm² to 10,000 μm². In some embodiments, the connection area of the first connection site and the second connection site may be in a range from 100 μm² to 10,000 μm². In some embodiments, the connection area of the first connection site and the second connection site may be in a range from 10 μm² to 100 μm².

In some embodiments, the aforementioned first electronic component 1210 may be prepared through a micro-nanofabrication process, and the overall thickness of the first electronic component 1210 may be in a micrometer or sub-micrometer level. In some embodiments, the first electronic component 1210 may include a plurality of mesh conductive structures (e.g., ranging from 2 to thousands of mesh conductive structures), each of which may serve as a connection terminal or a connection channel of the first electronic component 1210. In some embodiments, the plurality of mesh conductive structures may be distributed in an array. In some embodiments, the plurality of mesh conductive structures may be arranged in a linear array.

Referring to FIG. 13, in some embodiments, the first electronic component 1210 may further include a channel line 1213, and each of the plurality of mesh conductive structures may be connected to a corresponding circuit unit (e.g., an electrophysiological electrode, a signal acquisition circuit, etc.), respectively, via a corresponding channel line 1213.

Continuing to refer to FIG. 13, in some embodiments, the first electronic component may further include a first substrate 1212, and the aforementioned plurality of mesh conductive structures may be provided on a surface of the first substrate 1212. In some embodiments, the material of the first substrate 1212 may be or include a flexible insulating film material, such as dichloro-p-xylene dimer (Parylene-C), polyimide (PI), polydimethylsilocxane (PDMS), etc.

Similarly, in some embodiments, the second electronic component may include a second substrate. The thickness of the first substrate 1212 may be in a micrometer or sub-micrometer level, and based on similar principles illustrated above, when drying the first substrate 1212 and the second substrate, it may be possible to cause physical adsorption to form between the first substrate 1212 and the second substrate, so as to further strengthen the connection force between the first electronic component and the second electronic component.

Exemplarily, in some embodiments, the first electronic component 1210 may be prepared using a thin-film deposition technique, a micro-nanofabrication technique, and/or a CMOS integrated circuit technique, or one or more other fabrication techniques. Exemplarily, in some embodiments, the first electronic component 1210 may be prepared using a combination of a thin-film deposition technique, a photolithography technique, and a plasma etching technique. Specifically, in some embodiments, a polyamic acid solution may be spin-coated on the surface of a silicon wafer coated with a nickel sacrificial layer, followed by annealing under high vacuum to obtain an ultra-thin (e.g., about 500 nm) PI substrate (i.e., the first substrate). Then, a Cr/Au metal layer is obtained through orthogonal photolithography and magnetron sputtering on the PI substrate, and an upper PI layer is obtained using the same process as that of the PI substrate. On the upper PI layer, an Al metal mask layer is obtained through photolithography and magnetron sputtering, and then the meshing pattern (i.e., the mesh conductive structure) is obtained by using the reactive ion etching technique, and ferric chloride is used to etch the aluminum and the sacrificial layer of nickel, and after that, the first electronic component can be obtained by cleaning using deionized water.

It can be understood that in the embodiments provided herein, the size, shape, density and arrangement of the mesh conductive structure in the first electronic component may be various according to the actual situations (e.g., it may be adapted to the size, shape, density and arrangement of the second connection site in the second electronic component), which provides a relatively high degree of flexibility.

Using the above connection method provided in the embodiments of the present disclosure, the physically electrically conductive connection may be formed between the first connection site(s) and the second connection site(s). By re-creating a solution environment at the first connection site(s) and the second connection site(s), the first electronic component may be separated from the second electronic component after the physically electrically conductive connection is formed. Specifically, the first electronic component and the second electronic component may be separated by dripping a solution at the connection area between the first electronic component and the second electronic component, thereby realizing the reuse of the electronic components. It should be noted that the solution used for separating the first electronic component and the second electronic component may be the same or different from the solution used for connecting the first electronic component and the second electronic component.

In some embodiments, after separating the first connection site(s) of the first electronic component and the second connection site(s) of the second electronic component, the separated first connection site(s) and the second connection site(s) may be re-aligned and re-dried to reform a physically electrically conductive connection between the first connection site(s) and the second connection site(s).

In some embodiments, the first electronic component and/or the second electronic component may be reinforced after the first connection site(s) and the second connection site(s) form a physically electrically conductive connection, so as to further strengthen the connection force between the first electronic component and the second electronic component. For example, in some embodiments, the reinforced connection may be performed on at least one connection site of the first electronic component and the second electronic component. Exemplary reinforcement connection methods may include dispensing, soldering, etc.

FIGS. 14A and 14B are schematic diagrams illustrating an exemplary connection effect of a first electronic component and a printed circuit board according to some embodiments of the present disclosure.

Referring to FIGS. 14A-14B, in some embodiments, the second electronic component may be a printed circuit board, and the first connection site 1211 of the first electronic component and the second connection site 1221 of the second electronic component may be connected using the connection method described above. In some embodiments, according to experimental results, a connectivity rate of the first electronic component connected with the printed circuit board may reach more than 90%.

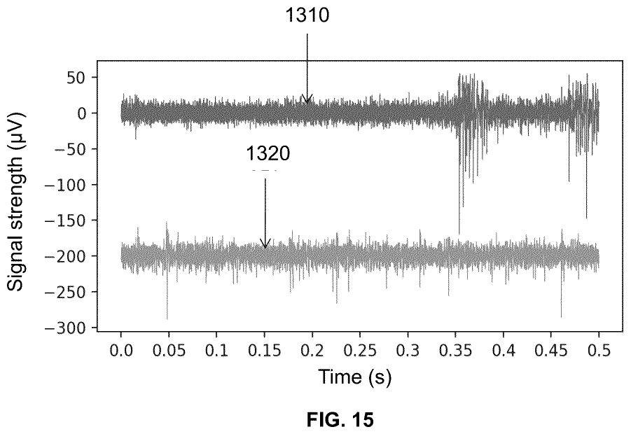

FIG. 15 is an exemplary waveform illustrating an application of connected electronic components in signal detection after the first electronic component is connected to the printed circuit board according to some embodiments of the present disclosure.

Referring to FIG. 15, waveform 1310 is a signal waveform diagram including neuronal action potentials obtained by applying the first electronic component to acute neural signal recording of a rat brain after the first electronic component is connected to the printed circuit board, and waveform 1320 is a signal waveform diagram including neuronal action potentials of the rat brain at the 25th week (after the start of the signal recording) obtained using the first electronic component after the first electronic component is connected to the printed circuit board. It can be seen from the test results shown in FIG. 15 that stable circuit conduction may be realized over a relatively long period of time after the first electronic component and the printed circuit board are connected using the connection method provided in the embodiments of the present disclosure.

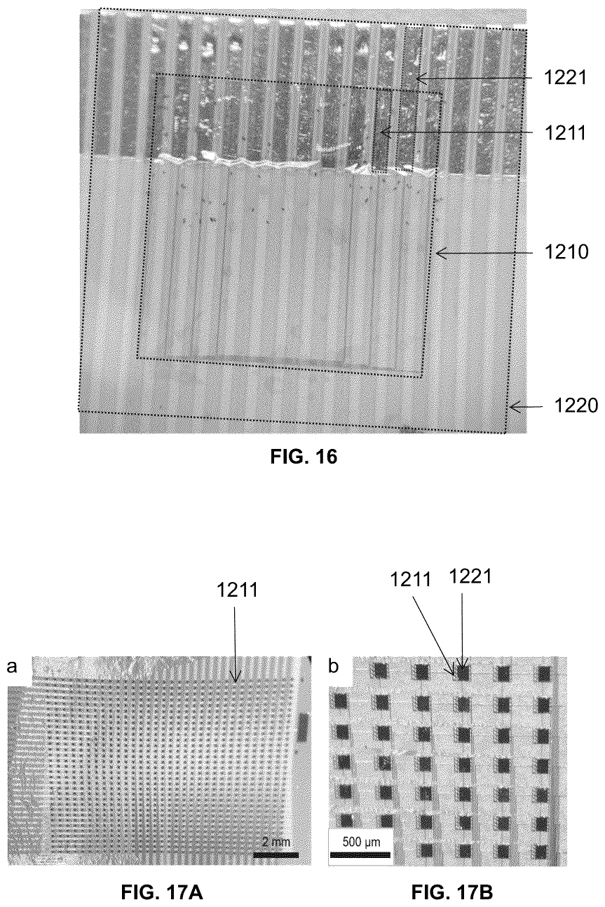

FIG. 16 is a schematic diagram illustrating an exemplary connection effect of a first electronic component and a flexible flat cable according to some embodiments of the present disclosure.

Referring to FIG. 16, in some embodiments, the second electronic component 1220 may be a flexible flat cable, the first electronic component 1210 may be provided on a surface of the second electronic component 1220, and the first connection site 1211 (the region (or a portion thereof) in the dashed box indicated by the serial number 1211 in FIG. 16) of the first electronic component 1210 and the second connection site 1221 (the region (or a portion thereof) in the dashed box indicated by the serial number 1221 in FIG. 16) of the second electronic component 1220 may be connected using the connection method described in the present disclosure. According to experimental results, if the second electronic component is a flexible flat cable, the connectivity rate of the first electronic component and the second electronic component may reach 100%. It should be noted that the connection effect shown in FIG. 16 is only an exemplary illustration, and in some embodiments, all or a portion of the first connection site 1211 of the first electronic component may partially overlap with a portion of the second connection site 1221 of the flexible flat cable; and in some embodiments, all or a portion of the first connection site 1211 of the first electronic component may cover the second connection site 1221 of the flexible flat cable.

FIGS. 17A and 17B are schematic diagrams illustrating an exemplary structure of the first electronic component connected with a flexible circuit board according to some embodiments of the present disclosure.

Referring to FIGS. 17A-17B, in some embodiments, the second electronic component may be a flexible circuit board, and the first connection site 1211 of the first electronic component and the second connection site 1221 of the second electronic component may be connected using the connection method described in the present disclosure. The second connection site 1221 of the second electronic component may be a connection point protruding from the flexible circuit board that is obtained by additional electrochemical deposition of a metal thereon. According to experimental results, if the second electronic component is a flexible circuit board, the connectivity rate of the first electronic component and the second electronic component may reach more than 95%.

Referring to FIGS. 17A-17B, in some embodiments, the first electronic component may include a plurality of first connection sites 1211, the second electronic component may include a plurality of second connection sites 1221, and the plurality of first connection sites 1211 and second connection sites 1221 may form a plurality of connection channels between the first electronic component and the second electronic component. According to experimental results, in some embodiments, after connecting the first electronic component and the second electronic component (e.g., a high-density flexible circuit board) using the connection method provided in the embodiments of the present disclosure, a connectivity density therebetween may reach 1542 channels/cm². In some embodiments, after connecting the first electronic component and the second electronic component (e.g., a high-density CMOS metal microelectrode array) using the connection method provided in the embodiments of the present disclosure, the connectivity density therebetween may reach 3265 channels/mm². It can be seen that the connection method provided in the embodiments of the present disclosure may achieve high connectivity and very high electrical connectivity density, which is beneficial for miniaturization of electronic component(s) and application of the electronic component(s) in integration application scenario(s).

According to the embodiments of the present disclosure: (1) by means of the connection method provided in the present disclosure, a high-throughput connection between electronic components can be realized without heating and/or pressurization, the connection method is simple and easy to operate, multiple channels can be connected in parallel using a single, one-touch, simple and fast operation, and the requirements for connection environment conditions are relatively low, which makes the connection method be applicable to various connection scenarios; (2) by means of the connection method provided in the present disclosure, it is possible to separate the physically electrically conductive connection formed between the first connection site(s) and the second connection site(s) by re-creating a solution environment at the first connection site(s) and the second connection site(s) after the first electronic component is connected to the second electronic component, to realize the reuse of the electronic component(s); (3) by means of the connection method provided in the present disclosure, a plurality of electronic components (such as printed circuit board(s), flexible circuit board(s), flexible flat cable(s), microprocessor chip(s), CMOS chip(s), integrated circuit chip(s), etc.) may be applied in a wide range of application scenarios, which provides a relatively high adaptability; (4) by means of the connection method provided in the present disclosure, it is beneficial to realize a high connectivity rate and a relatively high electrical connectivity density (the connectivity density may reach 3,265 channels/mm²), which is conducive to miniaturization of electronic component(s) and application of the electronic component(s) in integration application scenario(s).

It should be noted that different embodiments may produce different beneficial effects, and in different embodiments, the possible beneficial effects may be any one or a combination of the above, or any other beneficial effect that may be obtained.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A device, including:
a target electrode array having a plurality of electrode sites arranged circumferentially around a surface of the device and/or axially along the surface of the device, wherein the device is formed by scrolling a planar electrode array and/or attaching the planar electrode array around a carrier.

2. The device of claim 1, wherein the planar electrode array includes:
an electrode site part, the plurality of electrode sites of the target electrode array being arranged in the electrode site part;
a lead part configured to connect the electrode site part to an interface part;
the interface part configured to connect the device to a second device; and
a connection part for connection to the carrier.

3. The device of claim 2, wherein after the planar electrode array is scrolled and/or attached around the carrier, the electrode site part is exposed on a surface of the device, the lead part is embedded in an internal part of the device or exposed on a surface of the device, and the interface part is connected to the lead part.

4. (canceled)

5. The device of claim 2, wherein
the connection part includes a hole structure or a mesh structure, such that the carrier passes through the hole structure or the mesh structure to be fixed on the connection part, or
the carrier is bonded to the connection part of the planar electrode array via an adhesive material, or
the carrier is fixed on the connection part of the planar electrode array by putting the carrier into a groove on the planar electrode array.

6-9. (canceled)

10. The device of claim 2, wherein the electrode site part and the connection part are arranged on opposite sides of the planar electrode array.

11. The device of claim 1, wherein the carrier and the planar electrode array are configured as an integral piece.

12. The device of claim 1, wherein a side of the planar electrode array has a bevel edge structure, the bevel edge structure being configured to form a conical structure on the device by scrolling the planar electrode array.

13. (canceled)

14. The device of claim 1, wherein at least one of a substrate or an encapsulated layer of the planar electrode array is made of a flexible material.

15-19. (canceled)

20. The device of claim 1, wherein the carrier is configured to scroll the planar electrode array, improve a mechanical strength of the device, perform an optical stimulation or an electrical stimulation on a subject, or perform a delivery of drugs and/or reagents.

21. The device of claim 1, wherein the carrier includes at least one of a metal wire, a metal tube, a quartz wire, a quartz tube, an optical fiber, a polymer wire, a polymer tube, a combination of wire and tube, a ceramic wire, a graphene fiber, a carbon fiber, a stereoelectroencephalography (SEEG) electrode, or an intracranial pressure measuring device.

22-24. (canceled)

25. A method for preparing a device, comprising:
preparing a planar electrode array; and
scrolling the planar electrode array and/or attaching the planar electrode array around a carrier to form the device, the device having a columnar structure, wherein the scrolling the planar electrode array around a carrier to form the device comprises:
fixing the carrier on a first side of the planar electrode array;
scrolling the planar electrode array around the carrier; and
coating, in a last turn of the scrolling, an edge of a second side of the planar electrode array with an adhesive material, to fix the second side of the planar electrode array on the device.

26. The method of claim 25, wherein the preparing a planar electrode array comprises:
preparing a nickel-deposited silicon wafer by depositing nickel on a silicon wafer, wherein the nickel is determined as a sacrificial layer of the planar electrode array;
preparing a substrate of the planar electrode array by spin coating, heating, and curing at least one of polyamic acid, polydimethylsiloxane (PDMS), SU-8 photoresist, or by depositing Parylene-C, on the nickel-deposited silicon wafer;
preparing a conductive layer of the planar electrode array by depositing and patterning at least one of a metal, a carbon nanotube, a graphene, a carbon material, iridium oxide, titanium nitride, indium tin oxide, tantalum oxide, or a conductive polymer on the substrate of the planar electrode array; and
releasing the planar electrode array by etching the sacrificial layer from the silicon wafer using ferric chloride solution.

27. The method of claim 26, wherein after the preparing a conductive layer of the planar electrode array, the method further comprises:
preparing an encapsulated layer of the planar electrode array by spin coating, heating, and curing at least one of polyamic acid, PDMS, SU-8 photoresist, or by depositing Parylene-C, on the conductive layer of the planar electrode array;
preparing a mask by depositing and patterning aluminium on the encapsulated layer of the planar electrode array to obtain a masked encapsulated layer; and
obtaining a patterned electrode array by processing the masked encapsulated layer using reactive ion etching;
and the releasing the planar electrode array by etching the sacrificial layer from the silicon wafer using ferric chloride solution comprises:
releasing the planar electrode array by etching the mask and the sacrificial layer from the silicon wafer using the ferric chloride solution.

28-35. (canceled)

36. The method of claim 25, wherein the carrier is capable of being fully or partly removed from or left with the device.

37. (canceled)

38. The method of claim 25, wherein the device includes a first connection site, and the device is connected to a second device by:
aligning the first connection site of the device with a second connection site of the second device in a solution environment; and
drying the first connection site and the second connection site, such that a physically electrically conductive connection is formed between the aligned first connection site and second connection site.

39. A method for connecting electronic components, comprising:
aligning a first connection site of a first electronic component with a second connection site of a second electronic component in a solution environment; and
drying the first connection site and the second connection site, such that a physically electrically conductive connection is formed between the aligned first connection site and second connection site.

40. The method of claim 39, wherein
the first electronic component includes a flexible electronic component; and
the first connection site includes a first conductive structure.

41. (canceled)

42. The method of claim 39, wherein the first connection site has a mesh structure.

43. The method of claim 39, wherein
the second electronic component includes at least one of a printed circuit board, a flexible flat cable, a flexible circuit board, a micromachined chip, a complementary metal oxide semiconductor (CMOS) chip, an integrated circuit chip, or a CMOS metal microelectrode array; and
the second connection site includes a second conductive structure.

44-48. (canceled)

49. The method of claim 39, wherein the first electronic component includes a device, the device including:
a target electrode array having a plurality of electrode sites arranged circumferentially around a surface of the device and/or axially along the surface of the device, wherein the device is formed by scrolling a planar electrode array and/or attaching the planar electrode array around a carrier.

50. (canceled)

51. The device of claim 2, wherein the lead part is of a “U” shape.
